(19) United States
(12) Patent Application Publication
(10) Pub. No.: US 2024/0352529 A1
(43) Pub. Date: Oct. 24, 2024

(54) COLORECTAL CANCER SUBTYPE IDENTIFIER

(71) Applicant: Indivumed GmbH, Hamburg (DE)

(72) Inventors: Aslihan Ambeskovic, Rochester, NY (US); Karl Hartmut Land, Rochester, NY (US); Jonathan Woodsmith, Hamburg (DE)

(73) Assignee: Indivumed GmbH, Hamburg (DE)

(21) Appl. No.: 18/137,040

(22) Filed: Apr. 20, 2023

(51) Int. Cl. C12Q 1/6886

(52) U.S. Cl. CPC C12Q 1/6886; C12Q 2600/112; C12Q 2600/118; C12Q 2600/156

(57) ABSTRACT

The present invention relates to a colorectal cancer subtype identifier based on percent spliced-in (PSI) values from alternative splicing events. The invention further relates to a method for predicting the outcome for a subject suffering from colorectal cancer by using PSI values. The invention further relates to a kit for predicting the outcome for a subject suffering from colorectal cancer by PSI values. The invention further relates to a use of the PSI value as a marker of the outcome for a subject suffering from colorectal cancer. The invention further relates to method for treating a subject suffering from colorectal cancer using PSI values to obtain CMS likelihood on the outcome of the disease.

### FIELD

The present invention relates to a colorectal cancer subtype identifier based on percent spliced-in (PSI) values from alternative splicing events. The invention further relates to a method for predicting the outcome for a subject suffering from colorectal cancer by using PSI values. The invention further relates to a kit for predicting the outcome for a subject suffering from colorectal cancer by PSI values. The invention further relates to a use of the PSI value as a marker of the outcome for a subject suffering from colorectal cancer. The invention further relates to method for treating a subject suffering from colorectal cancer using PSI values to obtain CMS likelihood on the outcome of the disease.

### BACKGROUND OF THE INVENTION

Colorectal cancer (CRC) (also known as colon cancer or bowel cancer) is the 3rd most common cancer worldwide. There were more than 1.9 million new cases of colorectal cancer in 2020 and more than 500.000 patients per year are ultimately dying of the disease (Colorectal Cancer Facts & FIGS. 2020-2022, American Cancer Society, Atlanta, Georgia). Early-stage patients are typically treated with colon resection surgery, followed by radiation therapy or systemic chemotherapy, the therapy being based on macroscopic traits of the tumor and the tumor stage. The 5-year relapse-free survival rate is improved in some patients receiving chemotherapy after colon resection surgery, while this statistic is not improved in others.

Clinical experience and colon cancer research have led to the understanding that colon cancer is not one disease but occurs as several different subtypes. These subtypes are distinguished by distinct patterns of gene expression that influence or determine tumor biology. Many papers were published describing methods of classifying colon tumors into molecularly distinct subtypes, e.g., M. Pratap Singh et al., Genes & Diseases, 2021, 8, 133-145, G. Valenzuela et al., World J. Clin. Oncol., 2021, 12 (11), 1000-1008, D. G. Menter et al., Curr. Gastroenterol. Rep., 2019, 21 (5) 1-12, G. Martini et al., Ther. Adv. Med. Oncol., 2020, 12 (1), 1-18, E. Fontana et al., Annals of oncology, 2019, 30, 520-527. Recognizing similarities in many of these subtyping systems, a consortium of experts was formed to derive one subtyping system that inherits the best features from each system.

The consensus molecular subtype (CMS) classification system divides colon cancers into 4 distinct groups, called subtypes, each with distinct biological and molecular characteristics: CMS1, CMS2, CMS3 and CMS4. Gene expression profiles from tumors have been used to establish the subgroups. CMS1 (MSI Immune) tumors have microsatellite instability and are heavily infiltrated with immune cells. CMS2 (Canonical) is an epithelial subtype with significant upregulation of WNT and MYC downstream targets, and chromosomal instability (CIN) is a distinguishing feature of this subtype. CMS3 (Metabolic), like CMS2, has epithelial characteristics but exhibits less CIN. In addition, the CMS3 subtype is enriched for KRAS mutations and shows de-regulation of genes involved in metabolism. CMS4 (Mesenchymal) is a mesenchymal subtype and exhibits activation of pathways that regulate epithelial-mesenchymal transition (EMT) and stemness. The CMS subtype system is very likely to play a major role in colon cancer treatment selection.

Identification of colorectal cancer (CRC) molecular subtypes has prognostic and potentially diagnostic value for patients, yet reliable subtyping remains unavailable. Current consensus molecular subtype (CMS) classification in colorectal cancers is based on complex RNA expression patterns quantified at gene level.

WO2019173647 discloses a kit and a method of predicting or determining a consensus molecular subtype (CMS) of colorectal cancer in a human patient. The kit comprises a plurality of oligonucleotide primers configured to bind complementarily to respective portions of cDNA of at least twelve RNA transcripts from a colorectal cancer tissue sample and prime polymerase chain reaction of the cDNA, the at least twelve RNA transcripts comprising at least three RNA transcripts from each of four groups of genetic elements, each of the four groups of genetic elements defining a CMS gene expression profile specific for a different one of CMS1, CMS2, CMS3, and CMS4.

WO 2020206136 relates to a method of classifying a cancer status of colorectal cancer patients by
(a) obtaining a tumor sample from the subject;
(b) measuring an expression level of a plurality of genes in the tumor sample;
(c) generating an expression profile based on a comparison between the expression level of the plurality of genes in the sample from the subject and a corresponding expression level obtained from a reference sample derived from a different subject having a known cancer status; and
(d) categorizing the cancer status of the subject based on the expression profile.

WO2021061990 discloses a method of determining whether a subject has a CI subtype of colorectal cancer (CRC), wherein the expression level of at least one CI subtype-associated gene in the test sample is determined.

So far, the features of CMS and current CMS classification methods rely on expression patterns of hundreds of genes. Barriers to clinical application of these technologies include uncertainty in evaluation of samples one patient at a time which is commonly encountered in clinical environments, susceptibility of gene-level expression quantification to batch effects and the high cost associated with assessing gene expression levels of hundreds of genes.

Accordingly, there are several disadvantages in conventional consensus molecular subtype (CMS) determination for colorectal cancer tumors that can be addressed. Consequently, to determine consensus molecular subtype and the associated prognosis of colorectal tumors, there is an urgent need for a time and cost-effective clinical test.

Therefore, it is an object of the invention to provide a method for classifying molecular subtypes (CMS) for patients having colorectal cancer. It is further an object of the invention to provide a method for predicting the outcome for a subject suffering from a colorectal cancer.

Surprisingly, it was found that a colorectal cancer subtype identifier utilizing percent spliced-in (PSI) values derived from alternative splicing events is capable of classifying consensus molecular subtypes (CMS).

### SUMMARY OF THE INVENTION

The invention relates to a colorectal cancer subtype identifier, which classifies tumors by PSI values based on the occurrence of at least one alternative splicing event.

The invention further relates to a method for predicting the outcome for a subject suffering from colorectal cancer using PSI values based on the occurrence of at least one alternative splicing event comprising the steps:
a) collecting a sample from a tumor of a subject having cancer;
b) determining PSI values, in particular at least three, especially at least five PSI values;
c) converting PSI values to determine the disease outcome indication.

The invention further relates to a kit for predicting the outcome for a subject suffering from colorectal cancer using PSI values, in particular at least three, especially at least five PSI values, based on the occurrence of at least one alternative splicing event.

The invention further relates to a use of PSI values, in particular at least three, especially at least five PSI values, based on the occurrence of alternative splicing as a marker of the outcome for a subject suffering from colorectal cancer.

The invention further relates to a method for treating a subject suffering from colorectal cancer using PSI values based on the occurrence of alternative splicing comprising the steps:
d) collecting a sample from a tumor of a subject having cancer;
e) determining PSI values, in particular at least three, especially at least five PSI values;
f) conversion of PSI values to determine the disease outcome indication.

### DESCRIPTION OF THE DRAWINGS

The CMS identifier was constructed and validated using data from the colorectal cancer patients, in particular using detectable alternative splicing events (AS) present in 675 human colorectal cancers (data obtained from Indivumed Inc/GmbH, Indivumed cohort). CMS labels have been generated for the entire cohort using two different expression-based classifiers CMS Caller (Eide et al., Sci Rep. 2017; 7 (1):16618) and CMS Classifier (Guinney et al., Nat Med. 2015; 21 (11):1350-6). Unsupervised clustering was used to measure the strength of association between different categories of AS and CMS. To build a classifier, the ground-truth for CMS labels was derived from expression data quantified at gene-level. Feature selection was achieved through bootstrapping and L1-penalized estimation. The resulting feature space was used to train multiple logistic regression models using an Indivumed cohort of 300 CRCs. Model performance was evaluated on unseen CRCs from two independent sources. A colorectal cancer subtype identifier (CRCi) was developed based on 29 exon-skipping events that accurately classified unseen tumors (Indivumed AUC=0.95; TCGA AUC=0.93) and outperforms expression-based CMS classifiers.

FIG. 1: Exon-Skipping events having strong association with Colorectal Cancer Consensus Molecular Subtypes (CMS).
(A) Distribution of CMS labels identified by two independent expression-based methods for the Indivumed colorectal cancer cohort (n=429).
Including grayscale figures for B and C parts of the figure as well
(B) Strength of association between clusters emerging from NMF-based consensus clustering of percent spliced-in (PSI) values from alternative splicing events and CMS. The association strengths are determined by Cramer's V (a measure of effect size for the chi-square test of independence).
(C) Distribution of samples over NMF-based clusters ordered in relation to CMS (n=429).

FIG. 2: Performance of the colorectal cancer subtype identifier (CRCi). Receiver operating curves show the performance of CRCi on (A) the randomly selected test cohort (Indivumed, n=129) and (B) the validation cohort (TCGA, n=99).

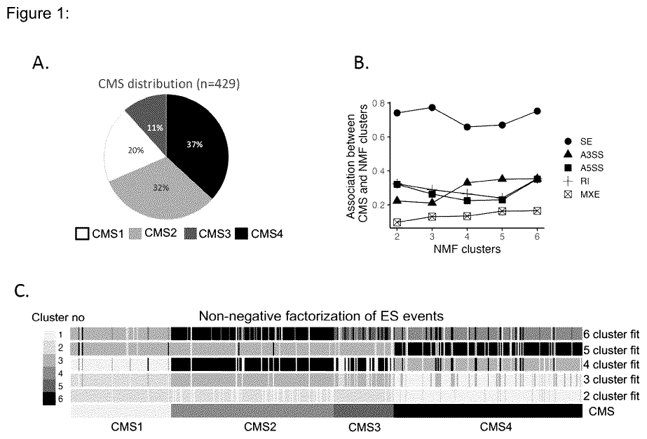

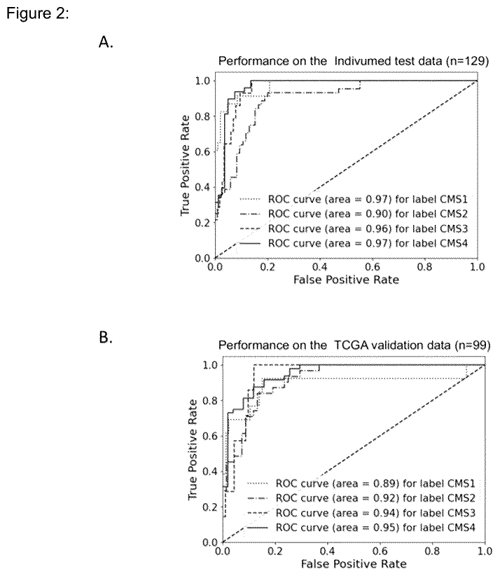

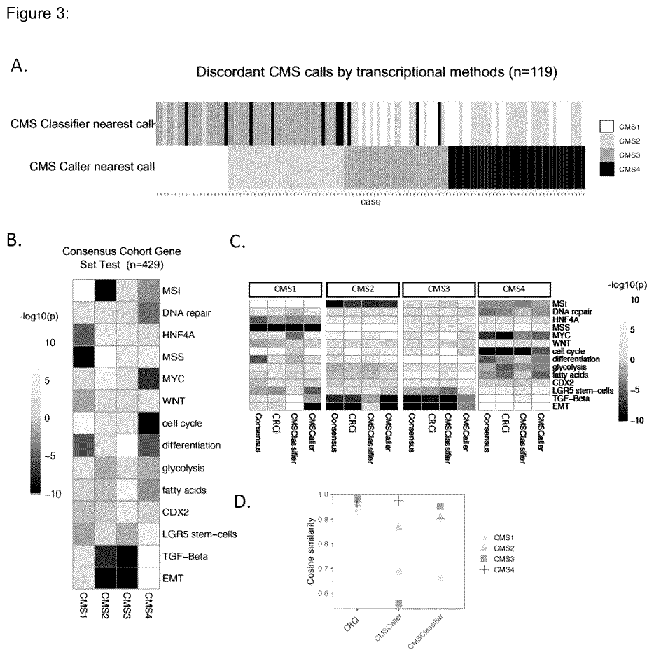

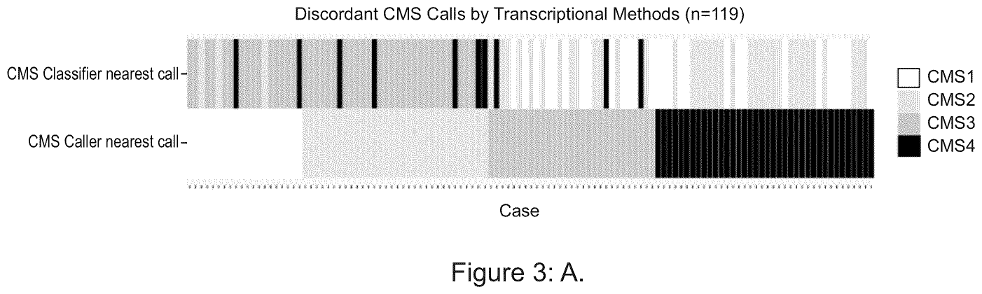

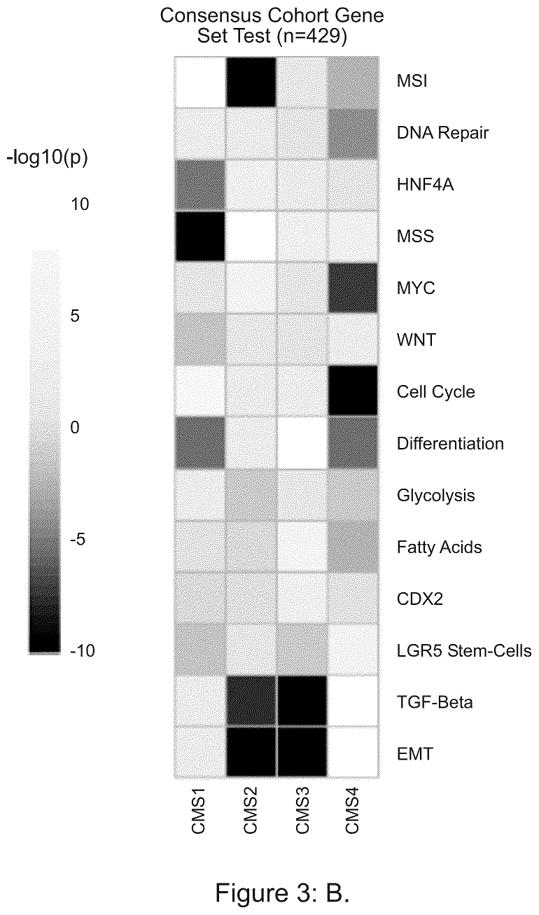

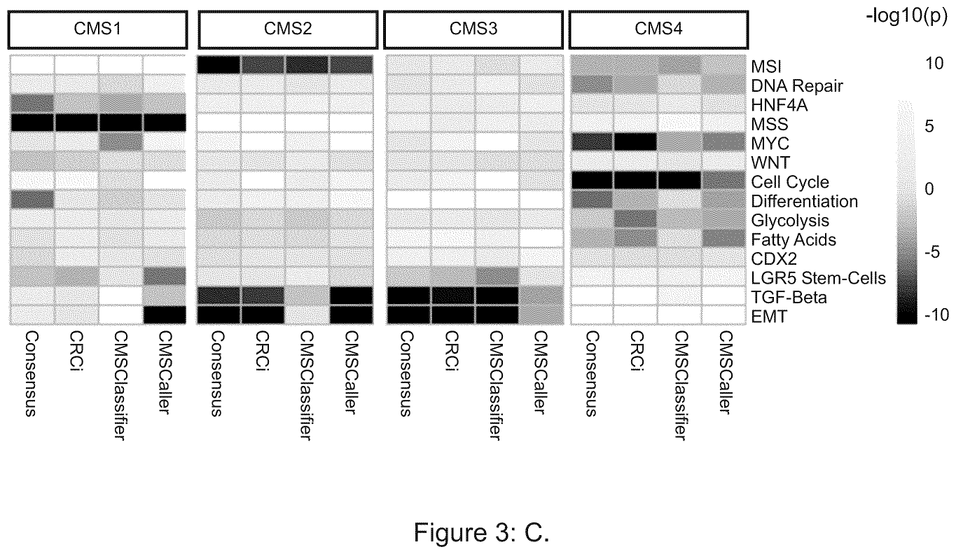

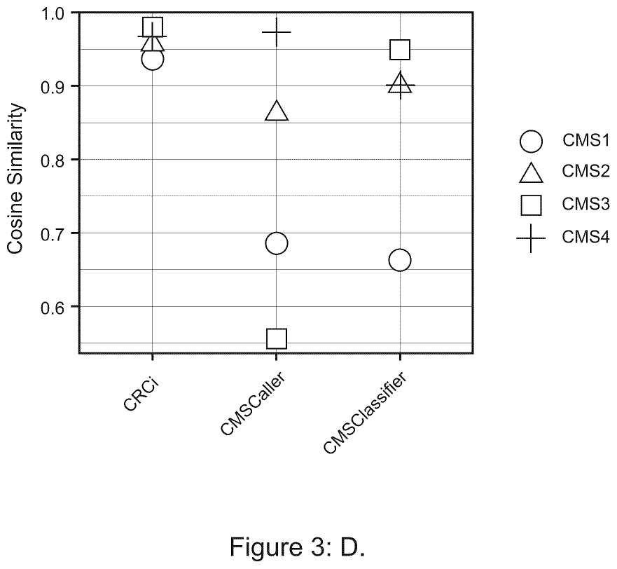

FIG. 3: CRCi outperforms expression-based methods in subtyping of discordant samples. 20
(A) Expression-based nearest CMS calls for the discordant tumor samples.
(B) Heatmap shows −log 10 transformed P-values from a competitive gene set test in the consensus cohort. Black indicates down-regulation and white indicates up-regulation.
(C) Heatmaps show −log 10 transformed P-values from a competitive gene set test comparing a consensus subtype or a subtype in the discordant cohort against all the other consensus subtypes.
(D) Cosine similarities calculated on transformed P-values from a competitive gene set test comparing a consensus subtype or a subtype in the discordant cohort against all the other consensus subtypes.

### DETAILED DESCRIPTION OF THE INVENTION

The colorectal cancer subtype identifier as well as the method according to the invention has the following advantages:
help to categorize patients into the subtypes CMS1, CMS2, CMS3 and CMS4 and decide on further treatment measures and/or surveillance as an alternative to nonintervention.
allow for identification of consensus molecular subtype of colorectal cancers by transforming PSI values from a small number of alternative splicing events per CMS which can be measured using RT-PCR, multiplex assays, or targeted sequencing.
PSI values provide the ratio of specific splice variants. The built-in normalization process to calculate PSI values makes this method more resistant to batch effects which can adversely affect quantification of gene-level expression.
provide evaluation of a single sample by providing four probabilities each representing the likelihood that the sample belongs to a particular subtype.
out-performs gene expression-based classifiers.

CMS1 (MSI Immune) tumors have microsatellite instability and are heavily infiltrated with immune cells. The pathway to carcinoma is characterized by: 1) proximal colon location, 2) high BRAFV600E mutation rate, 3) hypermethylation of CpG islands, which causes loss of tumor suppressor function (CpG island methylator phenotype [CIMP]), 4) an association with an impaired DNA mismatch repair (MMR) system, and 5) the infiltration of immunogenic lymphocytes in the tumor microenvironment. Patients with CMS1 tumors have a better prognosis compared to patients with CMS2, CMS3 or CMS4 tumors.

CMS2 (Canonical) is an epithelial subtype having activated WNT-β catenin and MYC signal transduction pathways wherein the chromosomal instability (CIN) is a distinguishing feature of this subtype. The tumors classified as CMS2 arises from the canonical adenoma-to-carcinoma sequence. CMS2 tumors are usually treated with standard adjuvant chemotherapy. Five-year overall survival for all stages of CMS2 are the highest of any subtype.

CMS3 (Metabolic) has epithelial characteristics but exhibits less CIN. In addition, the CMS3 subtype is enriched for KRAS mutations and shows de-regulation of genes involved in metabolism. CMS3 also has more MSI than CMS2 and 4. CMS3 appears the most similar to normal colon tissue at the gene expression level. Pathway analyses showed that CMS3 mRNA were enriched for 9 of 10 metabolic pathways investigated, including glutamine, fatty acid, and lysophospholipid metabolism.

CMS4 (Mesenchymal) is a mesenchymal subtype and exhibits activation of pathways that regulate epithelial-mesenchymal transition (EMT) and stemness. CMS4 tumors exhibit extremely low levels of hypermutation. CMS4 CRC displays a mesenchymal phenotype with gene signatures consistent with an activated stroma: angiogenesis, integrin binding to matrix proteins, TGFβ signalling characteristic of carcinoma-associated fibroblasts (CAF), and an inflammatory microenvironment with prominent innate immune cells. In contrast to the anti-tumor immune environment of CMS1 cancers, the CMS4 tumor microenvironment is pro-inflammatory, with the presence of Treg cells, T helper 17 cells, myleloid-derived suppressor cells, and tumor promoting macrophages. CMS4 cancers, often diagnosed at advanced stages, have a poor prognosis with the worst 5-year overall survival.

In the sense of the invention the term “subject” refers to any human or animal. A (non-human) animal includes all vertebrates, e.g., mammals and non-mammals, including cows, sheep, pigs, goats, horses, poultry, dogs, cats, non-human primates, rodents etc. In one embodiment, the subject is a human subject.

The term “marker” as used herein refers to a PSI value for determining the CMS1, CMS2, CMS3 and/or CMS4 groups.

In the sense of the invention the term “outcome” is the result of a treatment or series of treatments of a certain disease.

The outcome is determined at a point in time during or after the treatment on the basis of one criterion or a set of several criteria. Consideration of outcome helps to determine the effectiveness and appropriateness of medical interventions and to evaluate them in relation to alternatives, particularly nonintervention.

There are a number of definitions of the outcome of a disease, defined e.g., by using different endpoints. One way of determining the outcome is the “long-term” survival that refers to survival after diagnosis and/or initial treatment for a particular time period, e.g., for at least 3 years. Another way is the “Recurrence-Free Survival” (RFS) that refers to survival for a time period (usually in years) from diagnosis and/or initial treatment to cancer recurrence or death due to recurrence of cancer. Another way of determining is the “Overall Survival” (OS) that refers to the time (in years) from diagnosis and/or initial treatment to death from any cause. A further way is “Disease-Free Survival” (DFS) that refers to survival for a time period (usually in years) from diagnosis and/or initial treatment to first cancer recurrence or death from any cause.

“Outcome analyses” often focus on changes in quality of life; the respective preventive or therapeutic measures are thus to be evaluated in a more meaningful way for future subjects than is possible with the so-called surrogate markers, parameters or endpoints (these are measurable variables that are not directly relevant for the person concerned, such as measurement and laboratory values, tumor diameter).

Instead of surrogate parameters or an intuitive description of the case (“cured”/“not cured” or similar), a description of the overall situation should be as precisely defined as possible.

In a first embodiment the invention is directed to a colorectal cancer subtype identifier, which classifies tumors, based on PSI values associated with at least one alternative splicing event.

The designation of the reference genome is GRCh38, annotation is Gencode v28 (primary assembly).

The term “PSI”, the abbreviation of “percent spliced in”, refers to the exon-inclusion ratio. It is a known statistic value for measuring alternative splicing events. Alternative splicing allows a gene to be transcribed into multiple isoforms (or mRNA transcripts). PSI is defined as the ratio of the relative abundance of all isoforms containing a certain exon over the relative abundance of all isoforms of the gene containing the exon. In other words, the PSI value defines how often the exon occurs in all the isoforms of the gene that contains the exon. The PSI value of an alternative splicing event reflects the intensity/frequency of such events and has been widely used in detection of differentially spliced exons.

Alternative splicing (AS) events contribute to the complexity of gene expression patterns and can be categorized into five distinct types: (1) exon-skipping (ES), (2) intron retention (IR), (3) alternative 5′ splice (A5SS), (4) alternative 3′ splice (A3SS) and (5) mutually exclusive exon usage (MXE) events. These events are modulated in a tissue and cell type-specific manner and introduce qualitative changes into the existing RNA pool, adding an additional layer of biological information.

Preferably, the PSI value is based on at least 5, 10, 15, 20, 25, 30, 35, 40, 45, 50, 55, 60, 65, 70, 75, 80, 85, 90, 95, 100, 105, 110, 115, 120, 125, 130, 135, 141 alternative splicing events or a combination of these.

The alternative splicing event is preferably at least one exon skipping event.

Advantageously, colorectal cancer subtype identifier classifies the tumors as CMS1, CMS2, CMS3 or CMS4.

There are two types of alternative splicing patterns that can be used to identify colorectal cancer subtypes. The first type is a subtype specific alternative splicing event.

Preferably, for the identification of CMS1 at least 1, preferably at least 5, more preferably at least 10, in particular at least 20, especially at least 25, exon skipping events are present selected from ITGAE (ENSG00000083457.11) in exon with genomic coordinates chr17:3723287-3723383, CCNDBP1 (ENSG00000166946.13) in exon with genomic coordinates chr15:43194080-43194137, CPT1B (ENSG00000205560.12) in exon with genomic coordinates chr22:50573807-50573921, CDC16 (ENSG00000130177.14) in exon with genomic coordinates chr13:114236644-114236699, PTPN6 (ENSG00000111679.16) in exon with genomic coordinates chr12:6951458-6951520, PTPN6 (ENSG00000111679.16) in exon with genomic coordinates chr12:6951463-6951520, ACCS (ENSG00000110455.13) in exon with genomic coordinates chr11:44073446-44073517, EXOSC9 (ENSG00000123737.12) in exon with genomic coordinates chr4:121816143-121816194, EXOSC9 (ENSG00000123737.12) in exon with genomic coordinates chr4:121816368-121816447, ZNF611 (ENSG00000213020.9) in exon with genomic coordinates chr19:52707468-52707542, MRRF (ENSG00000148187.17) in exon with genomic coordinates chr9:122285779-122285946, NUP153 (ENSG00000124789.11) in exon with genomic coordinates chr6:17668974-17669028, WARS (ENSG00000140105.17) in exon with genomic coordinates chr14:100375282-100375350, WARS (ENSG00000140105.17) in exon with genomic coordinates chr14:100375282-100375403, WARS (ENSG00000140105.17) in exon with genomic coordinates chr14:100375282-100375406, D2HGDH (ENSG00000180902.17) in exon with genomic coordinates chr2:241748864-241749929, LUC7L (ENSG00000007392.16) in exon with genomic coordinates chr16:228332-228402, EPB41 (ENSG00000159023.20) in exon with genomic coordinates chr1:29058588-29058645, DGUOK (ENSG00000114956.19) in exon with genomic coordinates chr2:73938909-73939022, MDM4 (ENSG00000198625.12) in exon with genomic coordinates chr1:204537429-204537497, MDM4 (ENSG00000198625.12) in exon with genomic coordinates chr1:204537458-204537497, PTP4A2 (ENSG00000184007.19) in exon with genomic coordinates chr1:31915894-31915987, MKNK2 (ENSG00000099875.14) in exon with genomic coordinates chr19:2039630-2039856, CCDC112 (ENSG00000164221.12) in exon with genomic coordinates chr5:115269702-115269798, FRYL (ENSG00000075539.14) in exon with genomic coordinates chr4:48593929-48594016, CEP78 (ENSG00000148019.13) in exon with genomic coordinates chr9:78265858-78265906, FNBP1 (ENSG00000187239.16) in exon with genomic coordinates chr9:129923843-129923996, FNBP1 (ENSG00000187239.16) in exon with genomic coordinates chr9:129923843-129924026, ECT2 (ENSG00000114346.13) in exon with genomic coordinates chr3:172752149-172752472, ECT2 (ENSG00000114346.13) in exon with genomic coordinates chr3:172755482-172755575, ANKRD26 (ENSG00000107890.16) in exon with genomic coordinates chr10:27044156-27044190, ZMIZ2 (ENSG00000122515.14) in exon with genomic coordinates chr7:44760150-44760228, C6orf48 (ENSG00000204387.12) in exon with genomic coordinates chr6:31836423-31836517 and USPL1 (ENSG00000132952.11) in exon with genomic coordinates chr13:30621072-30621239.

In particular, for identification of CMS1 1, 2, 3, 4, 5, 6, 7, 8, 9 exon skipping events is/are present selected from ITGAE (ENSG00000083457.11) in exon with genomic coordinates chr17:3723287-3723383, MRRF (ENSG00000148187.17) in exon with genomic coordinates chr9:122285779-122285946, LUC7L (ENSG00000007392.16) in exon with genomic coordinates chr16:228332-228402, MDM4 (ENSG00000198625.12) in exon with genomic coordinates chr1:204537458-204537497, CCDC112 (ENSG00000164221.12) in exon with genomic coordinates chr5:115269702-115269798, FRYL (ENSG00000075539.14) in exon with genomic coordinates chr4:48593929-48594016, CEP78 (ENSG00000148019.13) in exon with genomic coordinates chr9:78265858-78265906, FNBP1 (ENSG00000187239.16) in exon with genomic coordinates chr9:129923843-129923996, ANKRD26 (ENSG00000107890.16) in exon with genomic coordinates chr10:27044156-27044190.

Preferably, for the identification of CMS2 at least 1, preferably at least 5, more preferably at least 10, in particular at least 15, exon skipping events are present selected from ZMIZ2 (ENSG00000122515.14) in exon with genomic coordinates chr7:44760150-44760228, C6orf48 (ENSG00000204387.12) in exon with genomic coordinates chr6:31836423-31836517, USPL1 (ENSG00000132952.11) in exon with genomic coordinates chr13:30621072-30621239, RBM39 (ENSG00000131051.22) in exon with genomic coordinates chr20:35740524-35740597, MIS12 (ENSG00000167842.15) in exon with genomic coordinates chr17:5488195-5488589, AFMID (ENSG00000183077.15) in exon with genomic coordinates chr17:78204655-78204741, MACROD1 (ENSG00000133315.10) in exon with genomic coordinates chr11:63998837-63998872, FN1 (ENSG00000115414.18) in exon with genomic coordinates chr2:215380810-215381080, WBP1 (ENSG00000239779.6) in exon with genomic coordinates chr2:74459477-74459562, XPO1 (ENSG00000082898.16) in exon with genomic coordinates chr2:61525269-61525333, PTPN18 (ENSG00000072135.12) in exon with genomic coordinates chr2:130359232-130359309, ARHGAP27 (ENSG00000159314.11) in exon with genomic coordinates chr17:45404268-45404334, C16orf13 (ENSG00000130731.15) in exon with genomic coordinates chr16:635280-635340, C16orf13 (ENSG00000130731.15) in exon with genomic coordinates chr16:635517-635774, C16orf13 (ENSG00000130731.15) in exon with genomic coordinates chr16:635611-635774.

In particular, for identification of CMS2 1, 2, 3, 4, 5 exon skipping events is/are present selected from USPL1 (ENSG00000132952.11) in exon with genomic coordinates chr13:30621072-30621239, MACROD1 (ENSG00000133315.10) in exon with genomic coordinates chr11:63998837-63998872, ENAH (ENSG00000154380.17) in exon with genomic coordinates chr1:225504990-225505053, FNIP1 (ENSG00000217128.11) in exon with genomic coordinates chr5:131710577-131710661 and SORBS1 (ENSG00000095637.21) in exon with genomic coordinates chr10:95414493-95414862.

Preferably, for the identification of CMS3 at least 1, preferably at least 5, more preferably at least 15, in particular at least 30, especially at least 41, exon skipping events are present selected from C16orf13 (ENSG00000130731.15) in exon with genomic coordinates chr16:635280-635340, C16orf13 (ENSG00000130731.15) in exon with genomic coordinates chr16:635517-635774, C16orf13 (ENSG00000130731.15) in exon with genomic coordinates chr16:635611-635774, AURKA (ENSG00000087586.17) in exon with genomic coordinates chr20:56388686-56388784, EPB41L3 (ENSG00000082397.17) in exon with genomic coordinates chr18:5394676-5394793, KALRN (ENSG00000160145.15) in exon with genomic coordinates chr3:124637207-124637303, ADAM15 (ENSG00000143537.13) in exon with genomic coordinates chr1:155061903-155061975, KRAS (ENSG00000133703.11) in exon with genomic coordinates chr12:25215436-25215560, SLC39A14 (ENSG00000104635.13) in exon with genomic coordinates chr8:22412036-22412206, MYO9A (ENSG00000066933.15) in exon with genomic coordinates chr15:71951776-71951896, TPM1 (ENSG00000140416.20) in exon with genomic coordinates chr15:63044026-63044152, TPM1 (ENSG00000140416.20) in exon with genomic coordinates chr15:63061197-63061273, TPM1 (ENSG00000140416.20) in exon with genomic coordinates chr15:63061712-63061788, MYO6 (ENSG00000196586.13) in exon with genomic coordinates chr6:75894813-75894840, MYO6 (ENSG00000196586.13) in exon with genomic coordinates chr6:75898372-75898411, KIAA1217 (ENSG00000120549.17) in exon with genomic coordinates chr10:24494499-24494604, KIAA1217 (ENSG00000120549.17) in exon with genomic coordinates chr10:24542692-24542770, KIAA1217 (ENSG00000120549.17) in exon with genomic coordinates chr10:24542882-24544481, GIT2 (ENSG00000139436.20) in exon with genomic coordinates chr12:109945259-109945349, MYL6 (ENSG00000092841.18) in exon with genomic coordinates chr12:56160625-56160670, CTNND1 (ENSG00000198561.13) in exon with genomic coordinates chr11:57789036-57789155, CTNND1 (ENSG00000198561.13) in exon with genomic coordinates chr11:57791384-57791673, CTNND1 (ENSG00000198561.13) in exon with genomic coordinates chr11:57791491-57791673, CD44 (ENSG00000026508.18) in exon with genomic coordinates chr11:35208104-35208206, CD44 (ENSG00000026508.18) in exon with genomic coordinates chr11:35211245-35211449, APBB2 (ENSG00000163697.16) in exon with genomic coordinates chr4:40935076-40935139, SEC31A (ENSG00000138674.16) in exon with genomic coordinates chr4:82830936-82830975, SEC31A (ENSG00000138674.16) in exon with genomic coordinates chr4:82830936-82830975, SEC31A (ENSG00000138674.16) in exon with genomic coordinates chr4:82842139-82842481, SEC31A (ENSG00000138674.16) in exon with genomic coordinates chr4:82842184-82842481, SYTL2 (ENSG00000137501.17) in exon with genomic coordinates chr11:85717482-85717530, MYOF (ENSG00000138119.16) in exon with genomic coordinates chr10:93392916-93392955, NAV2 (ENSG00000166833.19) in exon with genomic coordinates chr11:20051288-20051333, GAB1 (ENSG00000109458.8) in exon with genomic coordinates chr4:143434087-143434168, PLEKHM2 (ENSG00000116786.12) in exon with genomic coordinates chr1:15721328-15721388, CLSTN1 (ENSG00000171603.16) in exon with genomic coordinates chr1:9737497-9737554, CLSTN1 (ENSG00000171603.16) in exon with genomic coordinates chr1:9756480-9756510, GOLGA4 (ENSG00000144674.16) in exon with genomic coordinates chr3:37361242-37361305, PBX1 (ENSG00000185630.18) in exon with genomic coordinates chr1:164820071-164820184, FKBP14 (ENSG00000106080.10) in exon with genomic coordinates chr7:30020212-30020304 and XPO1 (ENSG00000082898.16) in exon with genomic coordinates chr2:61525269-61525333.

In particular, for identification of CMS3 1, 2, 3, 4, 5, 6, 7, 8, 9, 10, 11 exon skipping events is/are present selected from XPO1 (ENSG00000082898.16) in exon with genomic coordinates chr2:61525269-61525333, C16orf13 (ENSG00000130731.15) in exon with genomic coordinates chr16:635517-635774, PXN (ENSG00000089159.16) in exon with genomic coordinates chr12:120224642-120224727, ENAH (ENSG00000154380.17) in exon with genomic coordinates chr1:225504990-225505053, AURKA (ENSG00000087586.17) in exon with genomic coordinates chr20:56388686-56388784, EPB41L3 (ENSG00000082397.17) in exon with genomic coordinates chr18:5394676-5394793, KALRN (ENSG00000160145.15) in exon with genomic coordinates chr3:124637207-124637303, TPM1 (ENSG00000140416.20) in exon with genomic coordinates chr15:63061197-63061273, KIAA1217 (ENSG00000120549.17) in exon with genomic coordinates chr10:24542692-24542770, MYOF (ENSG00000138119.16) in exon with genomic coordinates chr10:93392916-93392955 and PBX1 (ENSG00000185630.18) in exon with genomic coordinates chr1:164820071-164820184.

Preferably, for the identification of CMS4 at least 1, preferably at least 5, more preferably at least 15, in particular at least 30, especially at least 55, exon skipping events are present selected from MYO9A (ENSG00000066933.15) in exon with genomic coordinates chr15:71951776-71951896, TPM1 (ENSG00000140416.20) in exon with genomic coordinates chr15:63044026-63044152, TPM1 (ENSG00000140416.20) in exon with genomic coordinates chr15:63061197-63061273, TPM1 (ENSG00000140416.20) in exon with genomic coordinates chr15:63061712-63061788, MYO6 (ENSG00000196586.13) in exon with genomic coordinates chr6:75894813-75894840, MYO6 (ENSG00000196586.13) in exon with genomic coordinates chr6:75898372-75898411, KIAA1217 (ENSG00000120549.17) in exon with genomic coordinates chr10:24494499-24494604, KIAA1217 (ENSG00000120549.17) in exon with genomic coordinates chr10:24542692-24542770, KIAA1217 (ENSG00000120549.17) in exon with genomic coordinates chr10:24542882-24544481, GIT2 (ENSG00000139436.20) in exon with genomic coordinates chr12:109945259-109945349, MYL6 (ENSG00000092841.18) in exon with genomic coordinates chr12:56160625-56160670, BPTF (ENSG00000171634.17) in exon with genomic coordinates chr17:67875555-67875744, MYH11 (ENSG00000133392.17) in exon with genomic coordinates chr16:15708802-15708841, NUMB (ENSG00000133961.20) in exon with genomic coordinates chr14:73279280-73279424, TEAD1 (ENSG00000187079.16) in exon with genomic coordinates chr11:12878888-12878900, SPAG9 (ENSG00000008294.20) in exon with genomic coordinates chr17:50975862-50975901, FAT1 (ENSG00000083857.13) in exon with genomic coordinates chr4:186590367-186590403, TNS1 (ENSG00000079308.17) in exon with genomic coordinates chr2:217830366-217830390, ESYT2 (ENSG00000117868.15) in exon with genomic coordinates chr7:158752780-158752843, SLMAP (ENSG00000163681.14) in exon with genomic coordinates chr3:57925844-57925934, AKAP9 (ENSG00000127914.16) in exon with genomic coordinates chr7:91992157-91992211, RUBCN (ENSG00000145016.15) in exon with genomic coordinates chr3:197691073-197691148, ATP2B4 (ENSG00000058668.14) in exon with genomic coordinates chr1:203733222-203733400, LRRFIP2 (ENSG00000093167.17) in exon with genomic coordinates chr3:37091466-37091538, TBC1D23 (ENSG00000036054.12) in exon with genomic coordinates chr3:100311832-100311877, EHBP1 (ENSG00000115504.14) in exon with genomic coordinates chr2:62987930-62988038, SLK (ENSG00000065613.13) in exon with genomic coordinates chr10:104010815-104010908, WDFY3 (ENSG00000163625.15) in exon with genomic coordinates chr4:84726860-84726911, SMARCC2 (ENSG00000139613.11) in exon with genomic coordinates chr12:56164302-56164368, KIF13A (ENSG00000137177.19) in exon with genomic coordinates chr6:17771113-17771218, MPRIP (ENSG00000133030.20) in exon with genomic coordinates chr17:17180606-17180669, LRRFIP1 (ENSG00000124831.18) in exon with genomic coordinates chr2:237769625-237769818, NIN (ENSG00000100503.23) in exon with genomic coordinates chr14:50756491-50758630, RPS24 (ENSG00000138326.19) in exon with genomic coordinates chr10:78040203-78040225, ACTN1 (ENSG00000072110.13) in exon with genomic coordinates chr14:68878988-68879069, CTNND1 (ENSG00000198561.13) in exon with genomic coordinates chr11:57789036-57789155, CTNND1 (ENSG00000198561.13) in exon with genomic coordinates chr11:57791384-57791673, CTNND1 (ENSG00000198561.13) in exon with genomic coordinates chr11:57791491-57791673, CD44 (ENSG00000026508.18) in exon with genomic coordinates chr11:35208104-35208206, CD44 (ENSG00000026508.18) in exon with genomic coordinates chr11:35211245-35211449, APBB2 (ENSG00000163697.16) in exon with genomic coordinates chr4:40935076-40935139, SEC31A (ENSG00000138674.16) in exon with genomic coordinates chr4:82830936-82830975, SEC31A (ENSG00000138674.16) in exon with genomic coordinates chr4:82830936-82830975, SEC31A (ENSG00000138674.16) in exon with genomic coordinates chr4:82842139-82842481, SEC31A (ENSG00000138674.16) in exon with genomic coordinates chr4:82842184-82842481, SYTL2 (ENSG00000137501.17) in exon with genomic coordinates chr11:85717482-85717530, MYOF (ENSG00000138119.16) in exon with genomic coordinates chr10:93392916-93392955, NAV2 (ENSG00000166833.19) in exon with genomic coordinates chr11:20051288-20051333, GAB1 (ENSG00000109458.8) in exon with genomic coordinates chr4:143434087-143434168, PLEKHM2 (ENSG00000116786.12) in exon with genomic coordinates chr1:15721328-15721388, CLSTN1 (ENSG00000171603.16) in exon with genomic coordinates chr1:9737497-9737554, CLSTN1 (ENSG00000171603.16) in exon with genomic coordinates chr1:9756480-9756510, GOLGA4 (ENSG00000144674.16) in exon with genomic coordinates chr3:37361242-37361305, PBX1 (ENSG00000185630.18) in exon with genomic coordinates chr1:164820071-164820184 and FKBP14 (ENSG00000106080.10) in exon with genomic coordinates chr7:30020212-30020304.

In particular, for identification of CMS4 1, 2, 3, 4, 5, 6, 7, 8 exon skipping events is/are present selected from ENAH (ENSG00000154380.17) in exon with genomic coordinates chr1:225504990-225505053, TPM1 (ENSG00000140416.20) in exon with genomic coordinates chr15:63061197-63061273, MYO6 (ENSG00000196586.13) in exon with genomic coordinates chr6:75898372-75898411, SLMAP (ENSG00000163681.14) in exon with genomic coordinates chr3:57925844-57925934, KIF13A (ENSG00000137177.19) in exon with genomic coordinates chr6:17771113-17771218, MPRIP (ENSG00000133030.20) in exon with genomic coordinates chr17:17180606-17180669, SEC31A (ENSG00000138674.16) in exon with genomic coordinates chr4:82842184-82842481 and MYOF (ENSG00000138119.16) in exon with genomic coordinates chr10:93392916-93392955.

The second type of exon-skipping events have complex PSI patterns that distinguish multiple classes simultaneously.

Preferably, for distinction of CMS2 and CMS3 from CMS1 and CMS4 at least 1, preferably at least 5, more preferably at least 10, in particular at least 20, especially at least 23, exon skipping events are present selected from FCGRT (ENSG00000104870.12) in exon with genomic coordinates chr19:49521694-49521813, DOCK6 (ENSG00000130158.13) in exon with genomic coordinates chr19:11229297-11229390, EXOC7 (ENSG00000182473.21) in exon with genomic coordinates chr17:76090328-76090397, BPTF (ENSG00000171634.17) in exon with genomic coordinates chr17:67875555-67875744, PXN (ENSG00000089159.16) in exon with genomic coordinates chr12:120224642-120224727, EXOC1 (ENSG00000090989.17) in exon with genomic coordinates chr4:55888887-55888932, PLOD2 (ENSG00000152952.11) in exon with genomic coordinates chr3:146077861-146077924, CD47 (ENSG00000196776.15) in exon with genomic coordinates chr3:108049618-108049651, CD47 (ENSG00000196776.15) in exon with genomic coordinates chr3:108050577-108050602, ARHGEF11 (ENSG00000132694.18) in exon with genomic coordinates chr1:156938417-156938513, PBRM1 (ENSG00000163939.18) in exon with genomic coordinates chr3:52558248-52558413, MAGI1 (ENSG00000151276.23) in exon with genomic coordinates chr3:65448021-65448057, MBNL1 (ENSG00000152601.17) in exon with genomic coordinates chr3:152446703-152446757, TNC (ENSG00000041982.15) in exon with genomic coordinates chr9:115064646-115064919, ENAH (ENSG00000154380.17) in exon with genomic coordinates chr1:225504990-225505053, BAZ2B (ENSG00000123636.17) in exon with genomic coordinates chr2:159397073-159397100, RAI14 (ENSG00000039560.13) in exon with genomic coordinates chr5:34813573-34813660, FNIP1 (ENSG00000217128.11) in exon with genomic coordinates chr5:131710577-131710661, MAP3K7 (ENSG00000135341.17) in exon with genomic coordinates chr6:90544551-90544632, ERBIN (ENSG00000112851.14) in exon with genomic coordinates chr5:66068876-66069020, SULF2 (ENSG00000196562.14) in exon with genomic coordinates chr20:47659398-47659452, SORBS1 (ENSG00000095637.21) in exon with genomic coordinates chr10:95414493-95414655 and SORBS1 (ENSG00000095637.21) in exon with genomic coordinates chr10:95414493-95414862.

Preferably, for distinction of CMS2 and CMS4 from CMS1 and CMS3 at least 1, preferably at least 5, more preferably at least 8, in particular at least 11, exon skipping events are present selected from SRSF6 (ENSG00000124193.14) in exon with genomic coordinates chr20:43459152-43459420, WASH3P (ENSG00000185596.16) in exon with genomic coordinates chr15:101972595-101972694, OPA1 (ENSG00000198836.9) in exon with genomic coordinates chr3:193626091-193626202, GOLGB1 (ENSG00000173230.15) in exon with genomic coordinates chr3:121719645-121719753, GOLGB1 (ENSG00000173230.15) in exon with genomic coordinates chr3:121719645-121719768, APLP2 (ENSG00000084234.16) in exon with genomic coordinates chr11:130137255-130137291, CPNE1 (ENSG00000214078.12) in exon with genomic coordinates chr20:35658929-35659014, WIPF1 (ENSG00000115935.17) in exon with genomic coordinates chr2:174597600-174597858, ATG9A (ENSG00000198925.11) in exon with genomic coordinates chr2:219228433-219228482, NUBP2 (ENSG00000095906.16) in exon with genomic coordinates chr16:1786737-1786955 and ANO1 (ENSG00000131620.17) in exon with genomic coordinates chr11:70111125-70111191.

A preferred embodiment is a colorectal cancer subtype identifier, wherein the alternative splicing is based on at least one exon skipping event on at least one, in particular at least 50, especially at least 116 genes selected from ITGAE (ENSG00000083457.11), CCNDBP1 (ENSG00000166946.13), CPT1B (ENSG00000205560.12), CDC16 (ENSG00000130177.14), PTPN6 (ENSG00000111679.16), ACCS (ENSG00000110455.13), EXOSC9 (ENSG00000123737.12), ZNF611 (ENSG00000213020.9), MRRF (ENSG00000148187.17), NUP153 (ENSG00000124789.11), WARS (ENSG00000140105.17), D2HGDH (ENSG00000180902.17), LUC7L (ENSG00000007392.16), EPB41 (ENSG00000159023.20), DGUOK (ENSG00000114956.19), MDM4 (ENSG00000198625.12), PTP4A2 (ENSG00000184007.19), MKNK2 (ENSG00000099875.14), CCDC112 (ENSG00000164221.12), FRYL (ENSG00000075539.14), CEP78 (ENSG00000148019.13), FNBP1 (ENSG00000187239.16), ECT2 (ENSG00000114346.13), ANKRD26 (ENSG00000107890.16), ZMIZ2 (ENSG00000122515.14), C6orf48 (ENSG00000204387.12), USPL1 (ENSG00000132952.11), RBM39 (ENSG00000131051.22), MIS12 (ENSG00000167842.15), AFMID (ENSG00000183077.15), MACROD1 (ENSG00000133315.10), FN1 (ENSG00000115414.18), WBP1 (ENSG00000239779.6), XPO1 (ENSG00000082898.16), PTPN18 (ENSG00000072135.12), ARHGAP27 (ENSG00000159314.11), C16orf13 (ENSG00000130731.15), FCGRT (ENSG00000104870.12), DOCK6 (ENSG00000130158.13), EXOC7 (ENSG00000182473.21), BPTF (ENSG00000171634.17), PXN (ENSG00000089159.16), EXOC1 (ENSG00000090989.17), PLOD2 (ENSG00000152952.11), CD47 (ENSG00000196776.15), ARHGEF11 (ENSG00000132694.18), PBRM1 (ENSG00000163939.18), MAGI1 (ENSG00000151276.23), MBNL1 (ENSG00000152601.17), TNC (ENSG00000041982.15), ENAH (ENSG00000154380.17), BAZ2B (ENSG00000123636.17), RAI14 (ENSG00000039560.13), FNIP1 (ENSG00000217128.11), MAP3K7 (ENSG00000135341.17), ERBIN (ENSG00000112851.14), SULF2 (ENSG00000196562.14), SORBS1 (ENSG00000095637.21), SRSF6 (ENSG00000124193.14), WASH3P (ENSG00000185596.16), OPA1 (ENSG00000198836.9), GOLGB1 (ENSG00000173230.15), APLP2 (ENSG00000084234.16), CPNE1 (ENSG00000214078.12), WIPF1 (ENSG00000115935.17), ATG9A (ENSG00000198925.11), NUBP2 (ENSG00000095906.16), ANO1 (ENSG00000131620.17), AURKA (ENSG00000087586.17), EPB41L3 (ENSG00000082397.17), KALRN (ENSG00000160145.15), ADAM15 (ENSG00000143537.13), KRAS (ENSG00000133703.11), SLC39A14 (ENSG00000104635.13), MYO9A (ENSG00000066933.15), TPM1 (ENSG00000140416.20), MYO6 (ENSG00000196586.13), KIAA1217 (ENSG00000120549.17), GIT2 (ENSG00000139436.20), MYL6 (ENSG00000092841.18), MYH11 (ENSG00000133392.17), NUMB (ENSG00000133961.20), TEAD1 (ENSG00000187079.16), SPAG9 (ENSG00000008294.20), FAT1 (ENSG00000083857.13), TNS1 (ENSG00000079308.17), ESYT2 (ENSG00000117868.15), SLMAP (ENSG00000163681.14), AKAP9 (ENSG00000127914.16), RUBCN (ENSG00000145016.15), ATP2B4 (ENSG00000058668.14), LRRFIP2 (ENSG00000093167.17), TBC1D23 (ENSG00000036054.12), EHBP1 (ENSG00000115504.14), SLK (ENSG00000065613.13), WDFY3 (ENSG00000163625.15), SMARCC2 (ENSG00000139613.11), KIF13A (ENSG00000137177.19), MPRIP (ENSG00000133030.20), LRRFIP1 (ENSG00000124831.18), NIN (ENSG00000100503.23), RPS24 (ENSG00000138326.19), ACTN1 (ENSG00000072110.13), CTNND1 (ENSG00000198561.13), CD44 (ENSG00000026508.18), APBB2 (ENSG00000163697.16), SEC31A (ENSG00000138674.16), SYTL2 (ENSG00000137501.17), MYOF (ENSG00000138119.16), NAV2 (ENSG00000166833.19), GAB1 (ENSG00000109458.8), PLEKHM2 (ENSG00000116786.12), CLSTN1 (ENSG00000171603.16), GOLGA4 (ENSG00000144674.16), PBX1 (ENSG00000185630.18) and FKBP14 (ENSG00000106080.10).

Another embodiment of the invention relates to a method for predicting the outcome for a subject suffering from colorectal cancer using PSI values based on the occurrence of at least one alternative splicing event, preferably, of at least one exon skipping event, comprising the steps:
a) collecting a sample from a tumor of a subject having cancer, preferably of epithelial cell origin;
b) determining the PSI value;
c) conversion of PSI values to determine the disease outcome indication.

In step a) a sample from a tumor in a subject having cancer is collected.

Suitable samples contain tissues or cells from the tumor of a subject having a cancer of epithelial cell origin. The sample preferably comprises a biopsy sample, such as a tumor biopsy, a primary tissue, a metastatic tissue. In particular, the specimen can be obtained by needle biopsy, image-guided biopsy, surgical (excisional) biopsy, shave/punch biopsy, endoscopic biopsy, laparoscopic biopsy and combinations thereof.

In step b) of the method according to the invention the PSI value is determined.

The PSI value represents the percentage of exon inclusion and is calculated/determined by dividing the number of reads from the upstream and downstream splice junctions of the alternative exon and/or from the alternative exon itself (referred to as inclusion reads) by the total number of reads from all splice junctions within the splice site, including the reads derived from the splice junction that connects the exon upstream of the alternative exon to the exon downstream of the alternative exon (referred as skipping reads) and inclusion reads. The effective length for the isoform is used for normalization of inclusion and skipping reads which is a mathematical transformation of sequencing read length and exon length.

PSI values, for example, can be estimated from RNA-Seq data using computational tools such as rMATS-turbo (https://github.com/Xinglab/rmats-turbo) or MISO (https://miso.readthedocs.io/en/fastmiso/).

There are several ways to convert the PSI values to determine the disease outcome.

One option is that the calculated PSI values are converted to CMS probabilities by mathematical transformation. Preferably, the calculated PSI values are converted to CMS probabilities estimates by mathematical transformation of the dot product between PSI values and their respective weights derived from the reference data set as follows:

PSI and θ respectively denote the vector representations for PSI values of a new sample and their associated weights with a consensus molecular subtype, as estimated from a penalized multinomial logistic regression model on the reference data set.

$$P S I = \begin{matrix}{PSI}_{1} \\ {PSI}_{2} \\ {PSI}_{n}\end{matrix}$$

$${θ}_{C M S \{ 1 , 2 , 3 , 4 \}} = \begin{matrix}{Weight}_{1 CMS \{ 1 , 2 , 3 , 4 \}} \\ {Weight}_{2 C M S \{ 1 , 2 , 3 , 4 \}} \\ {Weight}_{n C M S \{ 1 , 2 , 3 , 4 \}}\end{matrix}$$

z is the linear combination of the PSI values and their corresponding weights and β₀ is the intercept derived from the reference data set for each CMS:

$${z}_{C M S \{ 1 , 2 , 3 , 4 \}} = PSI \cdot {θ}_{C M S \{ 1 , 2 , 3 , 4 \}} = {\sum }_{i = 1}^{n} {PSI}_{i} {Weight}_{i C M S \{ 1 , 2 , 3 , 4 \}} + {β}_{{0}_{C M S \{ 1 , 2 , 3 , 4 \}}}$$

z is converted into a probability for each CMS using the logistic function where e is a mathematical constant (˜2.718):

$$P ( C M S \{ 1 , 2 , 3 , 4 \} ) = \frac{1}{1 + {e}^{- {Z}_{C M S \{ 1 , 2 , 3 , 4 \}}}}$$

Another possible approach for converting PSI values to CMS classification involves measuring the distance between a sample's PSI value and the median PSI for each CMS in the reference dataset. By ranking these distances, the CMS with the lowest distance can be assigned to the sample. For n number of PSI values, the CMS can be assigned as follows:

$${d}_{C M S 1} = {\sum }_{i = 1}^{n} \sqrt{{( m e d ( {PSI}_{{i}_{CMS 1 reference}} ) - {PSI}_{{i}_{sample}} )}^{2}}$$

$${d}_{C M S 2} = {\sum }_{i = 1}^{n} \sqrt{{( m e d ( {PSI}_{{i}_{CMS 2 reference}} ) - {PSI}_{{i}_{sample}} )}^{2}}$$

$${d}_{C M S 3} = {\sum }_{i = 1}^{n} \sqrt{{( m e d ( {PSI}_{{i}_{CMS 3 reference}} ) - {PSI}_{{i}_{sample}} )}^{2}}$$

$${d}_{C M S 4} = {\sum }_{i = 1}^{n} \sqrt{{( m e d ( {PSI}_{{i}_{CMS 4 reference}} ) - {PSI}_{{i}_{sample}} )}^{2}}$$

$$CMS = min ( {d}_{C M S 1} , {d}_{C M S 2} , {d}_{C M S 3} , {d}_{C M S 4} )$$

In step c) of the method according to the invention the conversion of PSI values to determine the disease outcome indication classifies the tumors as CMS1, CMS2, CMS3 or CMS4. Preferably, the conversion of PSI values to determine the disease outcome indication is used to decide, whether the subject is subjected to a treatment by anti-tumor therapy and/or surveillance of progress of the disease.

The identification of CMS1 is defined above.

The identification of CMS2 is defined above.

The identification of CMS3 is defined above.

The identification of CMS4 is defined above.

Preferably, the alternative splicing is based on at least one exon skipping event on at least one, in particular at least 50, especially at least 116 genes selected from ITGAE (ENSG00000083457.11), CCNDBP1 (ENSG00000166946.13), CPT1B (ENSG00000205560.12), CDC16 (ENSG00000130177.14), PTPN6 (ENSG00000111679.16), ACCS (ENSG00000110455.13), EXOSC9 (ENSG00000123737.12), ZNF611 (ENSG00000213020.9), MRRF (ENSG00000148187.17), NUP153 (ENSG00000124789.11), WARS (ENSG00000140105.17), D2HGDH (ENSG00000180902.17), LUC7L (ENSG00000007392.16), EPB41 (ENSG00000159023.20), DGUOK (ENSG00000114956.19), MDM4 (ENSG00000198625.12), PTP4A2 (ENSG00000184007.19), MKNK2 (ENSG00000099875.14), CCDC112 (ENSG00000164221.12), FRYL (ENSG00000075539.14), CEP78 (ENSG00000148019.13), FNBP1 (ENSG00000187239.16), ECT2 (ENSG00000114346.13), ANKRD26 (ENSG00000107890.16), ZMIZ2 (ENSG00000122515.14), C6orf48 (ENSG00000204387.12), USPL1 (ENSG00000132952.11), RBM39 (ENSG00000131051.22), MIS12 (ENSG00000167842.15), AFMID (ENSG00000183077.15), MACROD1 (ENSG00000133315.10), FN1 (ENSG00000115414.18), WBP1 (ENSG00000239779.6), XPO1 (ENSG00000082898.16), PTPN18 (ENSG00000072135.12), ARHGAP27 (ENSG00000159314.11), C16orf13 (ENSG00000130731.15), FCGRT (ENSG00000104870.12), DOCK6 (ENSG00000130158.13), EXOC7 (ENSG00000182473.21), BPTF (ENSG00000171634.17), PXN (ENSG00000089159.16), EXOC1 (ENSG00000090989.17), PLOD2 (ENSG00000152952.11), CD47 (ENSG00000196776.15), ARHGEF11 (ENSG00000132694.18), PBRM1 (ENSG00000163939.18), MAGI1 (ENSG00000151276.23), MBNL1 (ENSG00000152601.17), TNC (ENSG00000041982.15), ENAH (ENSG00000154380.17), BAZ2B (ENSG00000123636.17), RAI14 (ENSG00000039560.13), FNIP1 (ENSG00000217128.11), MAP3K7 (ENSG00000135341.17), ERBIN (ENSG00000112851.14), SULF2 (ENSG00000196562.14), SORBS1 (ENSG00000095637.21), SRSF6 (ENSG00000124193.14), WASH3P (ENSG00000185596.16), OPA1 (ENSG00000198836.9), GOLGB1 (ENSG00000173230.15), APLP2 (ENSG00000084234.16), CPNE1 (ENSG00000214078.12), WIPF1 (ENSG00000115935.17), ATG9A (ENSG00000198925.11), NUBP2 (ENSG00000095906.16), ANO1 (ENSG00000131620.17), AURKA (ENSG00000087586.17), EPB41L3 (ENSG00000082397.17), KALRN (ENSG00000160145.15), ADAM15 (ENSG00000143537.13), KRAS (ENSG00000133703.11), SLC39A14 (ENSG00000104635.13), MYO9A (ENSG00000066933.15), TPM1 (ENSG00000140416.20), MYO6 (ENSG00000196586.13), KIAA1217 (ENSG00000120549.17), GIT2 (ENSG00000139436.20), MYL6 (ENSG00000092841.18), MYH11 (ENSG00000133392.17), NUMB (ENSG00000133961.20), TEAD1 (ENSG00000187079.16), SPAG9 (ENSG00000008294.20), FAT1 (ENSG00000083857.13), TNS1 (ENSG00000079308.17), ESYT2 (ENSG00000117868.15), SLMAP (ENSG00000163681.14), AKAP9 (ENSG00000127914.16), RUBCN (ENSG00000145016.15), ATP2B4 (ENSG00000058668.14), LRRFIP2 (ENSG00000093167.17), TBC1D23 (ENSG00000036054.12), EHBP1 (ENSG00000115504.14), SLK (ENSG00000065613.13), WDFY3 (ENSG00000163625.15), SMARCC2 (ENSG00000139613.11), KIF13A (ENSG00000137177.19), MPRIP (ENSG00000133030.20), LRRFIP1 (ENSG00000124831.18), NIN (ENSG00000100503.23), RPS24 (ENSG00000138326.19), ACTN1 (ENSG00000072110.13), CTNND1 (ENSG00000198561.13), CD44 (ENSG00000026508.18), APBB2 (ENSG00000163697.16), SEC31A (ENSG00000138674.16), SYTL2 (ENSG00000137501.17), MYOF (ENSG00000138119.16), NAV2 (ENSG00000166833.19), GAB1 (ENSG00000109458.8), PLEKHM2 (ENSG00000116786.12), CLSTN1 (ENSG00000171603.16), GOLGA4 (ENSG00000144674.16), PBX1 (ENSG00000185630.18) and FKBP14 (ENSG00000106080.10).

Advantageously, in the method of the present invention it is possible to establish a rating scale by using the PSI value based on the occurrence of at least one alternative splicing event. Advantageously, this rating scale can be used to decide, whether the subject is likely to experience a poor outcome. In particular, the PSI value can be used to classify the tumors as CMS1, CMS2, CMS3 or CMS4. Further, it can be decided whether a subject should be subjected to a treatment by anti tumor therapy and/or surveillance of progress of the disease. This method allows a decision at a very early stage of the disease, where markers used in state of the art staging protocols (like e.g. the occurrence of other tumor markers or metastases), usually are not yet detectible.

A further embodiment of the invention relates to a kit for predicting the outcome for a subject suffering from colorectal cancer by PSI values based on the occurrence of at least one alternative splicing event. The kit comprises means for determining the PSI value. Preferably, the alternative splicing is based on at least one exon skipping event. In particular the at least one exon skipping event on at least one, in particular at least 50, especially at least 116 genes, selected from ITGAE (ENSG00000083457.11), CCNDBP1 (ENSG00000166946.13), CPT1B (ENSG00000205560.12), CDC16 (ENSG00000130177.14), PTPN6 (ENSG00000111679.16), ACCS (ENSG00000110455.13), EXOSC9 (ENSG00000123737.12), ZNF611 (ENSG00000213020.9), MRRF (ENSG00000148187.17), NUP153 (ENSG00000124789.11), WARS (ENSG00000140105.17), D2HGDH (ENSG00000180902.17), LUC7L (ENSG00000007392.16), EPB41 (ENSG00000159023.20), DGUOK (ENSG00000114956.19), MDM4 (ENSG00000198625.12), PTP4A2 (ENSG00000184007.19), MKNK2 (ENSG00000099875.14), CCDC112 (ENSG00000164221.12), FRYL (ENSG00000075539.14), CEP78 (ENSG00000148019.13), FNBP1 (ENSG00000187239.16), ECT2 (ENSG00000114346.13), ANKRD26 (ENSG00000107890.16), ZMIZ2 (ENSG00000122515.14), C6orf48 (ENSG00000204387.12), USPL1 (ENSG00000132952.11), RBM39 (ENSG00000131051.22), MIS12 (ENSG00000167842.15), AFMID (ENSG00000183077.15), MACROD1 (ENSG00000133315.10), FN1 (ENSG00000115414.18), WBP1 (ENSG00000239779.6), XPO1 (ENSG00000082898.16), PTPN18 (ENSG00000072135.12), ARHGAP27 (ENSG00000159314.11), C16orf13 (ENSG00000130731.15), FCGRT (ENSG00000104870.12), DOCK6 (ENSG00000130158.13), EXOC7 (ENSG00000182473.21), BPTF (ENSG00000171634.17), PXN (ENSG00000089159.16), EXOC1 (ENSG00000090989.17), PLOD2 (ENSG00000152952.11), CD47 (ENSG00000196776.15), ARHGEF11 (ENSG00000132694.18), PBRM1 (ENSG00000163939.18), MAGI1 (ENSG00000151276.23), MBNL1 (ENSG00000152601.17), TNC (ENSG00000041982.15), ENAH (ENSG00000154380.17), BAZ2B (ENSG00000123636.17), RAI14 (ENSG00000039560.13), FNIP1 (ENSG00000217128.11), MAP3K7 (ENSG00000135341.17), ERBIN (ENSG00000112851.14), SULF2 (ENSG00000196562.14), SORBS1 (ENSG00000095637.21), SRSF6 (ENSG00000124193.14), WASH3P (ENSG00000185596.16), OPA1 (ENSG00000198836.9), GOLGB1 (ENSG00000173230.15), APLP2 (ENSG00000084234.16), CPNE1 (ENSG00000214078.12), WIPF1 (ENSG00000115935.17), ATG9A (ENSG00000198925.11), NUBP2 (ENSG00000095906.16), ANO1 (ENSG00000131620.17), AURKA (ENSG00000087586.17), EPB41L3 (ENSG00000082397.17), KALRN (ENSG00000160145.15), ADAM15 (ENSG00000143537.13), KRAS (ENSG00000133703.11), SLC39A14 (ENSG00000104635.13), MYO9A (ENSG00000066933.15), TPM1 (ENSG00000140416.20), MYO6 (ENSG00000196586.13), KIAA1217 (ENSG00000120549.17), GIT2 (ENSG00000139436.20), MYL6 (ENSG00000092841.18), MYH11 (ENSG00000133392.17), NUMB (ENSG00000133961.20), TEAD1 (ENSG00000187079.16), SPAG9 (ENSG00000008294.20), FAT1 (ENSG00000083857.13), TNS1 (ENSG00000079308.17), ESYT2 (ENSG00000117868.15), SLMAP (ENSG00000163681.14), AKAP9 (ENSG00000127914.16), RUBCN (ENSG00000145016.15), ATP2B4 (ENSG00000058668.14), LRRFIP2 (ENSG00000093167.17), TBC1D23 (ENSG00000036054.12), EHBP1 (ENSG00000115504.14), SLK

The identification of CMS1 is defined above.

The identification of CMS2 is defined above.

The identification of CMS3 is defined above.

The identification of CMS4 is defined above.

Another embodiment of the invention is the use of the PSI value based on the occurrence of alternative splicing events as a marker of the outcome for a subject suffering from colorectal cancer. Preferably, the alternative splicing event is at least one exon skipping event.

The identification of CMS1 is defined above.

The identification of CMS2 is defined above.

The identification of CMS3 is defined above.

The identification of CMS4 is defined above.

Preferably, the alternative splicing is based on at least one exon skipping event on at least one, in particular at least 50, especially at least 116 genes, selected from ITGAE (ENSG00000083457.11), CCNDBP1 (ENSG00000166946.13), CPT1B (ENSG00000205560.12), CDC16 (ENSG00000130177.14), PTPN6 (ENSG00000111679.16), ACCS (ENSG00000110455.13), EXOSC9 (ENSG00000123737.12), ZNF611 (ENSG00000213020.9), MRRF (ENSG00000148187.17), NUP153 (ENSG00000124789.11), WARS (ENSG00000140105.17), D2HGDH (ENSG00000180902.17), LUC7L (ENSG00000007392.16), EPB41 (ENSG00000159023.20), DGUOK (ENSG00000114956.19), MDM4 (ENSG00000198625.12), PTP4A2 (ENSG00000184007.19), MKNK2 (ENSG00000099875.14), CCDC112 (ENSG00000164221.12), FRYL (ENSG00000075539.14), CEP78 (ENSG00000148019.13), FNBP1 (ENSG00000187239.16), ECT2 (ENSG00000114346.13), ANKRD26 (ENSG00000107890.16), ZMIZ2 (ENSG00000122515.14), C6orf48 (ENSG00000204387.12), USPL1 (ENSG00000132952.11), RBM39 (ENSG00000131051.22), MIS12 (ENSG00000167842.15), AFMID (ENSG00000183077.15), MACROD1 (ENSG00000133315.10), FN1 (ENSG00000115414.18), WBP1 (ENSG00000239779.6), XPO1 (ENSG00000082898.16), PTPN18 (ENSG00000072135.12), ARHGAP27 (ENSG00000159314.11), C16orf13 (ENSG00000130731.15), FCGRT (ENSG00000104870.12), DOCK6 (ENSG00000130158.13), EXOC7 (ENSG00000182473.21), BPTF (ENSG00000171634.17), PXN (ENSG00000089159.16), EXOC1 (ENSG00000090989.17), PLOD2 (ENSG00000152952.11), CD47 (ENSG00000196776.15), ARHGEF11 (ENSG00000132694.18), PBRM1 (ENSG00000163939.18), MAGI1 (ENSG00000151276.23), MBNL1 (ENSG00000152601.17), TNC (ENSG00000041982.15), ENAH (ENSG00000154380.17), BAZ2B (ENSG00000123636.17), RAI14 (ENSG00000039560.13), FNIP1 (ENSG00000217128.11), MAP3K7 (ENSG00000135341.17), ERBIN (ENSG00000112851.14), SULF2 (ENSG00000196562.14), SORBS1 (ENSG00000095637.21), SRSF6 (ENSG00000124193.14), WASH3P (ENSG00000185596.16), OPA1 (ENSG00000198836.9), GOLGB1 (ENSG00000173230.15), APLP2 (ENSG00000084234.16), CPNE1 (ENSG00000214078.12), WIPF1 (ENSG00000115935.17), ATG9A (ENSG00000198925.11), NUBP2 (ENSG00000095906.16), ANO1 (ENSG00000131620.17), AURKA (ENSG00000087586.17), EPB41L3 (ENSG00000082397.17), KALRN (ENSG00000160145.15), ADAM15 (ENSG00000143537.13), KRAS (ENSG00000133703.11), SLC39A14 (ENSG00000104635.13), MYO9A (ENSG00000066933.15), TPM1 (ENSG00000140416.20), MYO6 (ENSG00000196586.13), KIAA1217 (ENSG00000120549.17), GIT2 (ENSG00000139436.20), MYL6 (ENSG00000092841.18), MYH11 (ENSG00000133392.17), NUMB (ENSG00000133961.20), TEAD1 (ENSG00000187079.16), SPAG9 (ENSG00000008294.20), FAT1 (ENSG00000083857.13), TNS1 (ENSG00000079308.17), ESYT2 (ENSG00000117868.15), SLMAP (ENSG00000163681.14), AKAP9 (ENSG00000127914.16), RUBCN (ENSG00000145016.15), ATP2B4 (ENSG00000058668.14), LRRFIP2 (ENSG00000093167.17), TBC1D23 (ENSG00000036054.12), EHBP1 (ENSG00000115504.14), SLK (ENSG00000065613.13), WDFY3 (ENSG00000163625.15), SMARCC2 (ENSG00000139613.11), KIF13A (ENSG00000137177.19), MPRIP (ENSG00000133030.20), LRRFIP1 (ENSG00000124831.18), NIN (ENSG00000100503.23), RPS24 (ENSG00000138326.19), ACTN1 (ENSG00000072110.13), CTNND1 (ENSG00000198561.13), CD44 (ENSG00000026508.18), APBB2 (ENSG00000163697.16), SEC31A (ENSG00000138674.16), SYTL2 (ENSG00000137501.17), MYOF (ENSG00000138119.16), NAV2 (ENSG00000166833.19), GAB1 (ENSG00000109458.8), PLEKHM2 (ENSG00000116786.12), CLSTN1 (ENSG00000171603.16), GOLGA4 (ENSG00000144674.16), PBX1 (ENSG00000185630.18) and FKBP14 (ENSG00000106080.10).

Another embodiment of the invention is a method for treating a subject suffering from colorectal cancer using PSI values based on the occurrence of alternative splicing comprising the steps:
d) collecting a sample from a tumor of a subject having cancer, preferably of epithelial cell origin;
e) determining the PSI value;
f) conversion of PSI values to determine the disease outcome indication.

Preferably, the alternative splicing event is at least one exon skipping event.

The identification of CMS1 is defined above.

The identification of CMS2 is defined above.

The identification of CMS3 is defined above.

The identification of CMS4 is defined above.

Preferably, the alternative splicing is based on at least one exon skipping event on at least one, in particular at least 50, especially at least 116 genes, selected from ITGAE (ENSG00000083457.11), CCNDBP1 (ENSG00000166946.13), CPT1B (ENSG00000205560.12), CDC16 (ENSG00000130177.14), PTPN6 (ENSG00000111679.16), ACCS (ENSG00000110455.13), EXOSC9 (ENSG00000123737.12), ZNF611 (ENSG00000213020.9), MRRF (ENSG00000148187.17), NUP153 (ENSG00000124789.11), WARS (ENSG00000140105.17), D2HGDH (ENSG00000180902.17), LUC7L (ENSG00000007392.16), EPB41 (ENSG00000159023.20), DGUOK (ENSG00000114956.19), MDM4 (ENSG00000198625.12), PTP4A2 (ENSG00000184007.19), MKNK2 (ENSG00000099875.14), CCDC112 (ENSG00000164221.12), FRYL (ENSG00000075539.14), CEP78 (ENSG00000148019.13), FNBP1 (ENSG00000187239.16), ECT2 (ENSG00000114346.13), ANKRD26 (ENSG00000107890.16), ZMIZ2 (ENSG00000122515.14), C6orf48 (ENSG00000204387.12), USPL1 (ENSG00000132952.11), RBM39 (ENSG00000131051.22), MIS12 (ENSG00000167842.15), AFMID (ENSG00000183077.15), MACROD1 (ENSG00000133315.10), FN1 (ENSG00000115414.18), WBP1 (ENSG00000239779.6), XPO1 (ENSG00000082898.16), PTPN18 (ENSG00000072135.12), ARHGAP27 (ENSG00000159314.11), C16orf13 (ENSG00000130731.15), FCGRT (ENSG00000104870.12), DOCK6 (ENSG00000130158.13), EXOC7 (ENSG00000182473.21), BPTF (ENSG00000171634.17), PXN (ENSG00000089159.16), EXOC1 (ENSG00000090989.17), PLOD2 (ENSG00000152952.11), CD47 (ENSG00000196776.15), ARHGEF11 (ENSG00000132694.18), PBRM1 (ENSG00000163939.18), MAGI1 (ENSG00000151276.23), MBNL1 (ENSG00000152601.17), TNC (ENSG00000041982.15), ENAH (ENSG00000154380.17), BAZ2B (ENSG00000123636.17), RAI14 (ENSG00000039560.13), FNIP1 (ENSG00000217128.11), MAP3K7 (ENSG00000135341.17), ERBIN (ENSG00000112851.14), SULF2 (ENSG00000196562.14), SORBS1 (ENSG00000095637.21), SRSF6 (ENSG00000124193.14), WASH3P (ENSG00000185596.16), OPA1 (ENSG00000198836.9), GOLGB1 (ENSG00000173230.15), APLP2 (ENSG00000084234.16), CPNE1 (ENSG00000214078.12), WIPF1 (ENSG00000115935.17), ATG9A (ENSG00000198925.11), NUBP2 (ENSG00000095906.16), ANO1 (ENSG00000131620.17), AURKA (ENSG00000087586.17), EPB41L3 (ENSG00000082397.17), KALRN (ENSG00000160145.15), ADAM15 (ENSG00000143537.13), KRAS (ENSG00000133703.11), SLC39A14 (ENSG00000104635.13), MYO9A (ENSG00000066933.15), TPM1 (ENSG00000140416.20), MYO6 (ENSG00000196586.13), KIAA1217 (ENSG00000120549.17), GIT2 (ENSG00000139436.20), MYL6 (ENSG00000092841.18), MYH11 (ENSG00000133392.17), NUMB (ENSG00000133961.20), TEAD1 (ENSG00000187079.16), SPAG9 (ENSG00000008294.20), FAT1 (ENSG00000083857.13), TNS1 (ENSG00000079308.17), ESYT2 (ENSG00000117868.15), SLMAP (ENSG00000163681.14), AKAP9 (ENSG00000127914.16), RUBCN (ENSG00000145016.15), ATP2B4 (ENSG00000058668.14), LRRFIP2 (ENSG00000093167.17), TBC1D23 (ENSG00000036054.12), EHBP1 (ENSG00000115504.14), SLK (ENSG00000065613.13), WDFY3 (ENSG00000163625.15), SMARCC2 (ENSG00000139613.11), KIF13A (ENSG00000137177.19), MPRIP (ENSG00000133030.20), LRRFIP1 (ENSG00000124831.18), NIN (ENSG00000100503.23), RPS24 (ENSG00000138326.19), ACTN1 (ENSG00000072110.13), CTNND1 (ENSG00000198561.13), CD44 (ENSG00000026508.18), APBB2 (ENSG00000163697.16), SEC31A (ENSG00000138674.16), SYTL2 (ENSG00000137501.17), MYOF (ENSG00000138119.16), NAV2 (ENSG00000166833.19), GAB1 (ENSG00000109458.8), PLEKHM2 (ENSG00000116786.12), CLSTN1 (ENSG00000171603.16), GOLGA4 (ENSG00000144674.16), PBX1 (ENSG00000185630.18) and FKBP14 (ENSG00000106080.10).

Advantageously, in the method of the present invention it is possible to establish a rating scale by using the PSI value based on the occurrence of at least one alternative splicing event. Advantageously, this rating scale can be used to decide, whether the subject is likely to experience a poor outcome. In particular, the PSI values can be used to classify the tumors as CMS1, CMS2, CMS3 or CMS4. Further, it can be decided whether a subject should be subjected to a treatment by anti tumor therapy and/or surveillance of progress of the disease. This method allows a decision at a very early stage of the disease, where markers used in state of the art staging protocols (like e.g. the occurrence of other tumor markers or metastases), usually are not yet detectible.

### EXEMPLARY EMBODIMENTS

1. A colorectal cancer subtype identifier, which classifies tumors by PSI values based on the occurrence of at least one alternative splicing event.
2. The colorectal cancer subtype identifier according to embodiment 1, wherein the alternative splicing event is at least one exon skipping event.
3. The colorectal cancer subtype identifier according to any one of the preceding embodiments, for classifying the tumors as CMS1, CMS2, CMS3 or CMS4.
4. The colorectal cancer subtype identifier according to any one of the embodiments 1 to 3, wherein for the identification of CMS1 at least one exon skipping event is present selected from ITGAE in exon with genomic coordinates chr17:3723287-3723383, CCNDBP1 in exon with genomic coordinates chr15:43194080-43194137, CPT1B in exon with genomic coordinates chr22:50573807-50573921, CDC16 in exon with genomic coordinates chr13:114236644-114236699, PTPN6 in exon with genomic coordinates chr12:6951458-6951520, PTPN6 in exon with genomic coordinates chr12:6951463-6951520, ACCS in exon with genomic coordinates chr11:44073446-44073517, EXOSC9 in exon with genomic coordinates chr4:121816143-121816194, EXOSC9 in exon with genomic coordinates chr4:121816368-121816447, ZNF611 in exon with genomic coordinates chr19:52707468-52707542, MRRF in exon with genomic coordinates chr9:122285779-122285946, NUP153 in exon with genomic coordinates chr6:17668974-17669028, WARS in exon with genomic coordinates chr14:100375282-100375350, WARS in exon with genomic coordinates chr14:100375282-100375403, WARS in exon with genomic coordinates chr14:100375282-100375406, D2HGDH in exon with genomic coordinates chr2:241748864-241749929, LUC7L in exon with genomic coordinates chr16:228332-228402, EPB41 in exon with genomic coordinates chr1:29058588-29058645, DGUOK in exon with genomic coordinates chr2:73938909-73939022, MDM4 in exon with genomic coordinates chr1:204537429-204537497, MDM4 in exon with genomic coordinates chr1:204537458-204537497, PTP4A2 in exon with genomic coordinates chr1:31915894-31915987, MKNK2 in exon with genomic coordinates chr19:2039630-2039856, CCDC112 in exon with genomic coordinates chr5:115269702-115269798, FRYL in exon with genomic coordinates chr4:48593929-48594016, CEP78 in exon with genomic coordinates chr9:78265858-78265906, FNBP1 in exon with genomic coordinates chr9:129923843-129923996, FNBP1 in exon with genomic coordinates chr9:129923843-129924026, ECT2 in exon with genomic coordinates chr3:172752149-172752472, ECT2 in exon with genomic coordinates chr3:172755482-172755575, ANKRD26 in exon with genomic coordinates chr10:27044156-27044190, ZMIZ2 in exon with genomic coordinates chr7:44760150-44760228, C6orf48 in exon with genomic coordinates chr6:31836423-31836517 and USPL1 in exon with genomic coordinates chr13:30621072-30621239.
5. The colorectal cancer subtype identifier according to any one of embodiments 1 to 3, wherein for the identification of CMS2 at least one exon skipping event is present selected from ZMIZ2 in exon with genomic coordinates chr7:44760150-44760228, C6orf48 in exon with genomic coordinates chr6:31836423-31836517, USPL1 in exon with genomic coordinates chr13:30621072-30621239, RBM39 in exon with genomic coordinates chr20:35740524-35740597, MIS12 in exon with genomic coordinates chr17:5488195-5488589, AFMID in exon with genomic coordinates chr17:78204655-78204741, MACROD1 in exon with genomic coordinates chr11:63998837-63998872, FN1 in exon with genomic coordinates chr2:215380810-215381080, WBP1 in exon with genomic coordinates chr2:74459477-74459562, XPO1 in exon with genomic coordinates chr2:61525269-61525333, PTPN18 in exon with genomic coordinates chr2:130359232-130359309, ARHGAP27 in exon with genomic coordinates chr17:45404268-45404334, C16orf13 in exon with genomic coordinates chr16:635280-635340, C16orf13 in exon with genomic coordinates chr16:635517-635774 and C16orf13 in exon with genomic coordinates chr16:635611-635774.
6. The colorectal cancer subtype identifier according to any one of embodiment 1 to 3, wherein for the identification of CMS3 at least one exon skipping event is present selected from C16orf13 in exon with genomic coordinates chr16:635280-635340, C16orf13 in exon with genomic coordinates chr16:635517-635774, C16orf13 in exon with genomic coordinates chr16:635611-635774, AURKA in exon with genomic coordinates chr20:56388686-56388784, EPB41L3 in exon with genomic coordinates chr18:5394676-5394793, KALRN in exon with genomic coordinates chr3:124637207-124637303, ADAM15 in exon with genomic coordinates chr1:155061903-155061975, KRAS in exon with genomic coordinates chr12:25215436-25215560, SLC39A14 in exon with genomic coordinates chr8:22412036-22412206, MYO9A in exon with genomic coordinates chr15:71951776-71951896, TPM1 in exon with genomic coordinates chr15:63044026-63044152, TPM1 in exon with genomic coordinates chr15:63061197-63061273, TPM1 in exon with genomic coordinates chr15:63061712-63061788, MYO6 in exon with genomic coordinates chr6:75894813-75894840, MYO6 in exon with genomic coordinates chr6:75898372-75898411, KIAA1217 in exon with genomic coordinates chr10:24494499-24494604, KIAA1217 in exon with genomic coordinates chr10:24542692-24542770, KIAA1217 in exon with genomic coordinates chr10:24542882-24544481, GIT2 in exon with genomic coordinates chr12:109945259-109945349, MYL6 in exon with genomic coordinates chr12:56160625-56160670, CTNND1 in exon with genomic coordinates chr11:57789036-57789155, CTNND1 in exon with genomic coordinates chr11:57791384-57791673, CTNND1 in exon with genomic coordinates chr11:57791491-57791673, CD44 in exon with genomic coordinates chr11:35208104-35208206, CD44 in exon with genomic coordinates chr11:35211245-35211449, APBB2 in exon with genomic coordinates chr4:40935076-40935139, SEC31A in exon with genomic coordinates chr4:82830936-82830975, SEC31A in exon with genomic coordinates chr4:82830936-82830975, SEC31A in exon with genomic coordinates chr4:82842139-82842481, SEC31A in exon with genomic coordinates chr4:82842184-82842481, SYTL2 in exon with genomic coordinates chr11:85717482-85717530, MYOF in exon with genomic coordinates chr10:93392916-93392955, NAV2 in exon with genomic coordinates chr11:20051288-20051333, GAB1 in exon with genomic coordinates chr4:143434087-143434168, PLEKHM2 in exon with genomic coordinates chr1:15721328-15721388, CLSTN1 in exon with genomic coordinates chr1:9737497-9737554, CLSTN1 in exon with genomic coordinates chr1:9756480-9756510, GOLGA4 in exon with genomic coordinates chr3:37361242-37361305, PBX1 in exon with genomic coordinates chr1:164820071-164820184, FKBP14 in exon with genomic coordinates chr7:30020212-30020304 and XPO1 in exon with genomic coordinates chr2:61525269-61525333.
7. The colorectal cancer subtype identifier according to any one of embodiments 1 to 3, wherein for the identification of CMS4 at least one exon skipping event is present selected from MYO9A in exon with genomic coordinates chr15:71951776-71951896, TPM1 in exon with genomic coordinates chr15:63044026-63044152, TPM1 in exon with genomic coordinates chr15:63061197-63061273, TPM1 in exon with genomic coordinates chr15:63061712-63061788, MYO6 in exon with genomic coordinates chr6:75894813-75894840, MYO6 in exon with genomic coordinates chr6:75898372-75898411, KIAA1217 in exon with genomic coordinates chr10:24494499-24494604, KIAA1217 in exon with genomic coordinates chr10:24542692-24542770, KIAA1217 in exon with genomic coordinates chr10:24542882-24544481, GIT2 in exon with genomic coordinates chr12:109945259-109945349, MYL6 in exon with genomic coordinates chr12:56160625-56160670, BPTF in exon with genomic coordinates chr17:67875555-67875744, MYH11 in exon with genomic coordinates chr16:15708802-15708841, NUMB in exon with genomic coordinates chr14:73279280-73279424, TEAD1 in exon with genomic coordinates chr11:12878888-12878900, SPAG9 in exon with genomic coordinates chr17:50975862-50975901, FAT1 in exon with genomic coordinates chr4:186590367-186590403, TNS1 in exon with genomic coordinates chr2:217830366-217830390, ESYT2 in exon with genomic coordinates chr7:158752780-158752843, SLMAP in exon with genomic coordinates chr3:57925844-57925934, AKAP9 in exon with genomic coordinates chr7:91992157-91992211, RUBCN in exon with genomic coordinates chr3:197691073-197691148, ATP2B4 in exon with genomic coordinates chr1:203733222-203733400, LRRFIP2 in exon with genomic coordinates chr3:37091466-37091538, TBC1D23 in exon with genomic coordinates chr3:100311832-100311877, EHBP1 in exon with genomic coordinates chr2:62987930-62988038, SLK in exon with genomic coordinates chr10:104010815-104010908, WDFY3 in exon with genomic coordinates chr4:84726860-84726911, SMARCC2 in exon with genomic coordinates chr12:56164302-56164368, KIF13A in exon with genomic coordinates chr6:17771113-17771218, MPRIP in exon with genomic coordinates chr17:17180606-17180669, LRRFIP1 in exon with genomic coordinates chr2:237769625-237769818, NIN in exon with genomic coordinates chr14:50756491-50758630, RPS24 in exon with genomic coordinates chr10:78040203-78040225, ACTN1 in exon with genomic coordinates chr14:68878988-68879069, CTNND1 in exon with genomic coordinates chr11:57789036-57789155, CTNND1 in exon with genomic coordinates chr11:57791384-57791673, CTNND1 in exon with genomic coordinates chr11:57791491-57791673, CD44 in exon with genomic coordinates chr11:35208104-35208206, CD44 in exon with genomic coordinates chr11:35211245-35211449, APBB2 in exon with genomic coordinates chr4:40935076-40935139, SEC31A in exon with genomic coordinates chr4:82830936-82830975, SEC31A in exon with genomic coordinates chr4:82830936-82830975, SEC31A in exon with genomic coordinates chr4:82842139-82842481, SEC31A in exon with genomic coordinates chr4:82842184-82842481, SYTL2 in exon with genomic coordinates chr11:85717482-85717530, MYOF in exon with genomic coordinates chr10:93392916-93392955, NAV2 in exon with genomic coordinates chr11:20051288-20051333, GAB1 in exon with genomic coordinates chr4:143434087-143434168, PLEKHM2 in exon with genomic coordinates chr1:15721328-15721388, CLSTN1 in exon with genomic coordinates chr1:9737497-9737554, CLSTN1 in exon with genomic coordinates chr1:9756480-9756510, GOLGA4 in exon with genomic coordinates chr3:37361242-37361305, PBX1 in exon with genomic coordinates chr1:164820071-164820184 and FKBP14 in exon with genomic coordinates chr7:30020212-30020304.
8. The colorectal cancer subtype identifier according to any one of the preceding embodiments, wherein for distinction of CMS2 and CMS3 from CMS1 and CMS4, at least one exon skipping events is present selected from FCGRT in exon with genomic coordinates chr19:49521694-49521813, DOCK6 in exon with genomic coordinates chr19:11229297-11229390, EXOC7 in exon with genomic coordinates chr17:76090328-76090397, BPTF in exon with genomic coordinates chr17:67875555-67875744, PXN in exon with genomic coordinates chr12:120224642-120224727, EXOC1 in exon with genomic coordinates chr4:55888887-55888932, PLOD2 in exon with genomic coordinates chr3:146077861-146077924, CD47 in exon with genomic coordinates chr3:108049618-108049651, CD47 in exon with genomic coordinates chr3:108050577-108050602, ARHGEF11 in exon with genomic coordinates chr1:156938417-156938513, PBRM1 in exon with genomic coordinates chr3:52558248-52558413, MAGI1 in exon with genomic coordinates chr3:65448021-65448057, MBNL1 in exon with genomic coordinates chr3:152446703-152446757, TNC in exon with genomic coordinates chr9:115064646-115064919, ENAH in exon with genomic coordinates chr1:225504990-225505053, BAZ2B in exon with genomic coordinates chr2:159397073-159397100, RAI14 in exon with genomic coordinates chr5:34813573-34813660, FNIP1 in exon with genomic coordinates chr5:131710577-131710661, MAP3K7 in exon with genomic coordinates chr6:90544551-90544632, ERBIN in exon with genomic coordinates chr5:66068876-66069020, SULF2 in exon with genomic coordinates chr20:47659398-47659452, SORBS1 in exon with genomic coordinates chr10:95414493-95414655 and SORBS1 in exon with genomic coordinates chr10:95414493-95414862.
9. The colorectal cancer subtype identifier according to any one of the preceding embodiments, wherein for distinction of CMS2 and CMS4 from CMS1 and CMS3, at least one exon skipping event is present selected from SRSF6 in exon with genomic coordinates chr20:43459152-43459420, WASH3P in exon with genomic coordinates chr15:101972595-101972694, OPA1 in exon with genomic coordinates chr3:193626091-193626202, GOLGB1 in exon with genomic coordinates chr3:121719645-121719753, GOLGB1 in exon with genomic coordinates chr3:121719645-121719768, APLP2 in exon with genomic coordinates chr11:130137255-130137291, CPNE1 in exon with genomic coordinates chr20:35658929-35659014, WIPF1 in exon with genomic coordinates chr2:174597600-174597858, ATG9A in exon with genomic coordinates chr2:219228433-219228482, NUBP2 in exon with genomic coordinates chr16:1786737-1786955 and ANO1 in exon with genomic coordinates chr11:70111125-70111191.
10. The colorectal cancer subtype identifier according to any one of the preceding embodiments, wherein the alternative splicing event is based on at least one exon skipping event on at least one gene selected from ITGAE, CCNDBP1, CPT1B, CDC16, PTPN6, ACCS, EXOSC9, ZNF611, MRRF, NUP153, WARS, D2HGDH, LUC7L, EPB41, DGUOK, MDM4, PTP4A2, MKNK2, CCDC112, FRYL, CEP78, FNBP1, ECT2, ANKRD26, ZMIZ2, C6orf48, USPL1, RBM39, MIS12, AFMID, MACROD1, FN1, WBP1, XPO1, PTPN18, ARHGAP27, C16orf13, FCGRT, DOCK6, EXOC7, BPTF, PXN, EXOC1, PLOD2, CD47, ARHGEF11, PBRM1, MAGI1, MBNL1, TNC, ENAH, BAZ2B, RAI14, FNIP1, MAP3K7, ERBIN, SULF2, SORBS1, SRSF6, WASH3P, OPA1, GOLGB1, APLP2, CPNE1, WIPF1, ATG9A, NUBP2, ANO1, AURKA, EPB41L3, KALRN, ADAM15, KRAS, SLC39A14, MYO9A, TPM1, MYO6, KIAA1217, GIT2, MYL6, MYH11, NUMB, TEAD1, SPAG9, FAT1, TNS1, ESYT2, SLMAP, AKAP9, RUBCN, ATP2B4, LRRFIP2, TBC1D23, EHBP1, SLK, WDFY3, SMARCC2, KIF13A, MPRIP, LRRFIP1, NIN, RPS24, ACTN1, CTNND1, CD44, APBB2, SEC31A, SYTL2, MYOF, NAV2, GAB1, PLEKHM2, CLSTN1, GOLGA4, PBX1 and FKBP14.
11. A method for predicting the outcome for a subject suffering from colorectal cancer using PSI values based on the occurrence of at least one alternative splicing event comprising the steps:
a) collecting a sample from a tumor of a subject having cancer;
b) determining the PSI value;
c) conversion of PSI values to determine the disease outcome indication.
12. The method of embodiment 11, wherein the alternative splicing event is at least one exon skipping event.
13. The method of embodiment 12, wherein the at least one exon skipping event is at least one exon skipping event on at least one gene selected from ITGAE, CCNDBP1, CPT1B, CDC16, PTPN6, ACCS, EXOSC9, ZNF611, MRRF, NUP153, WARS, D2HGDH, LUC7L, EPB41, DGUOK, MDM4, PTP4A2, MKNK2, CCDC112, FRYL, CEP78, FNBP1, ECT2, ANKRD26, ZMIZ2, C6orf48, USPL1, RBM39, MIS12, AFMID, MACROD1, FN1, WBP1, XPO1, PTPN18, ARHGAP27, C16orf13, FCGRT, DOCK6, EXOC7, BPTF, PXN, EXOC1, PLOD2, CD47, ARHGEF11, PBRM1, MAGI1, MBNL1, TNC, ENAH, BAZ2B, RAI14, FNIP1, MAP3K7, ERBIN, SULF2, SORBS1, SRSF6, WASH3P, OPA1, GOLGB1, APLP2, CPNE1, WIPF1, ATG9A, NUBP2, ANO1, AURKA, EPB41L3, KALRN, ADAM15, KRAS, SLC39A14, MYO9A, TPM1, MYO6, KIAA1217, GIT2, MYL6, MYH11, NUMB, TEAD1, SPAG9, FAT1, TNS1, ESYT2, SLMAP, AKAP9, RUBCN, ATP2B4, LRRFIP2, TBC1D23, EHBP1, SLK, WDFY3, SMARCC2, KIF13A, MPRIP, LRRFIP1, NIN, RPS24, ACTN1, CTNND1, CD44, APBB2, SEC31A, SYTL2, MYOF, NAV2, GAB1, PLEKHM2, CLSTN1, GOLGA4, PBX1 and FKBP14.
14. The method of embodiment 11, wherein the conversion of PSI values to determine the disease outcome indication classifies the tumors as CMS1, CMS2, CMS3 or CMS4.
15. The method of embodiment 11, wherein the conversion of PSI values to determine the disease outcome indication is used to decide, whether the subject is subjected to a treatment by anti-tumor therapy and/or surveillance of progress of the disease.
16. A kit for predicting the outcome for a subject suffering from colorectal cancer using PSI values based on the occurrence of at least one alternative splicing event, preferably at least one exon skipping event on at least one gene, selected from ITGAE, CCNDBP1, CPT1B, CDC16, PTPN6, ACCS, EXOSC9, ZNF611, MRRF, NUP153, WARS, D2HGDH, LUC7L, EPB41, DGUOK, MDM4, PTP4A2, MKNK2, CCDC112, FRYL, CEP78, FNBP1, ECT2, ANKRD26, ZMIZ2, C6orf48, USPL1, RBM39, MIS12, AFMID, MACROD1, FN1, WBP1, XPO1, PTPN18, ARHGAP27, C16orf13, FCGRT, DOCK6, EXOC7, BPTF, PXN, EXOC1, PLOD2, CD47, ARHGEF11, PBRM1, MAGI1, MBNL1, TNC, ENAH, BAZ2B, RAI14, FNIP1, MAP3K7, ERBIN, SULF2, SORBS1, SRSF6, WASH3P, OPA1, GOLGB1, APLP2, CPNE1, WIPF1, ATG9A, NUBP2, ANO1, AURKA, EPB41L3, KALRN, ADAM15, KRAS, SLC39A14, MYO9A, TPM1, MYO6, KIAA1217, GIT2, MYL6, MYH11, NUMB, TEAD1, SPAG9, FAT1, TNS1, ESYT2, SLMAP, AKAP9, RUBCN, ATP2B4, LRRFIP2, TBC1D23, EHBP1, SLK, WDFY3, SMARCC2, KIF13A, MPRIP, LRRFIP1, NIN, RPS24, ACTN1, CTNND1, CD44, APBB2, SEC31A, SYTL2, MYOF, NAV2, GAB1, PLEKHM2, CLSTN1, GOLGA4, PBX1 and FKBP14, comprising means for determining the PSI value.
17. A use of at least one PSI value based on the occurrence of alternative splicing as a marker of the outcome for a subject suffering from colorectal cancer.
18. A method for treating a subject suffering from colorectal cancer using PSI values based on the occurrence of alternative splicing comprising the steps:
d) collecting a sample from a tumor of a subject having cancer;
e) determining at least on PSI value;
f) conversion of PSI values to determine the disease outcome indication.
19. The method according to embodiment 18, wherein the conversion of PSI values to determine the disease outcome indication classifies the tumors as CMS1, CMS2, CMS3 or CMS4.
20. The method according to embodiments 11 or 18, wherein the subject is a human.

The invention will be illustrated further with reference to the examples that follow, without restricting the scope to the specific embodiments described. The invention includes all combinations of described and especially of preferred features that do not exclude each other.

1. A colorectal cancer subtype identifier, which classifies tumors by PSI values based on the occurrence of at least one alternative splicing event.

2. The colorectal cancer subtype identifier according to claim 1, wherein the alternative splicing event is at least one exon skipping event.

3. The colorectal cancer subtype identifier according to claim 1, for classifying the tumors as CMS1, CMS2, CMS3 or CMS4.

4. The colorectal cancer subtype identifier according to claim 3, wherein for the identification of CMS1 at least one exon skipping event is present selected from ITGAE in exon with genomic coordinates chr17:3723287-3723383, CCNDBP1 in exon with genomic coordinates chr15:43194080-43194137, CPT1B in exon with genomic coordinates chr22:50573807-50573921, CDC16 in exon with genomic coordinates chr13:114236644-114236699, PTPN6 in exon with genomic coordinates chr12:6951458-6951520, PTPN6 in exon with genomic coordinates chr12:6951463-6951520, ACCS in exon with genomic coordinates chr11:44073446-44073517, EXOSC9 in exon with genomic coordinates chr4:121816143-121816194, EXOSC9 in exon with genomic coordinates chr4:121816368-121816447, ZNF611 in exon with genomic coordinates chr19:52707468-52707542, MRRF in exon with genomic coordinates chr9:122285779-122285946, NUP153 in exon with genomic coordinates chr6:17668974-17669028, WARS in exon with genomic coordinates chr14:100375282-100375350, WARS in exon with genomic coordinates chr14:100375282-100375403, WARS in exon with genomic coordinates chr14:100375282-100375406, D2HGDH in exon with genomic coordinates chr2:241748864-241749929, LUC7L in exon with genomic coordinates chr16:228332-228402, EPB41 in exon with genomic coordinates chr1:29058588-29058645, DGUOK in exon with genomic coordinates chr2:73938909-73939022, MDM4 in exon with genomic coordinates chr1:204537429-204537497, MDM4 in exon with genomic coordinates chr1:204537458-204537497, PTP4A2 in exon with genomic coordinates chr1:31915894-31915987, MKNK2 in exon with genomic coordinates chr19:2039630-2039856, CCDC112 in exon with genomic coordinates chr5:115269702-115269798, FRYL in exon with genomic coordinates chr4:48593929-48594016, CEP78 in exon with genomic coordinates chr9:78265858-78265906, FNBP1 in exon with genomic coordinates chr9:129923843-129923996, FNBP1 in exon with genomic coordinates chr9:129923843-129924026, ECT2 in exon with genomic coordinates chr3:172752149-172752472, ECT2 in exon with genomic coordinates chr3:172755482-172755575, ANKRD26 in exon with genomic coordinates chr10:27044156-27044190, ZMIZ2 in exon with genomic coordinates chr7:44760150-44760228, C6orf48 in exon with genomic coordinates chr6:31836423-31836517 and USPL1 in exon with genomic coordinates chr13:30621072-30621239.

5. The colorectal cancer subtype identifier according to claim 3, wherein for the identification of CMS2 at least one exon skipping event is present selected from ZMIZ2 in exon with genomic coordinates chr7:44760150-44760228, C6orf48 in exon with genomic coordinates chr6:31836423-31836517, USPL1 in exon with genomic coordinates chr13:30621072-30621239, RBM39 in exon with genomic coordinates chr20:35740524-35740597, MIS12 in exon with genomic coordinates chr17:5488195-5488589, AFMID in exon with genomic coordinates chr17:78204655-78204741, MACROD1 in exon with genomic coordinates chr11:63998837-63998872, FN1 in exon with genomic coordinates chr2:215380810-215381080, WBP1 in exon with genomic coordinates chr2:74459477-74459562, XPO1 in exon with genomic coordinates chr2:61525269-61525333, PTPN18 in exon with genomic coordinates chr2:130359232-130359309, ARHGAP27 in exon with genomic coordinates chr17:45404268-45404334, C16orf13 in exon with genomic coordinates chr16:635280-635340, C16orf13 in exon with genomic coordinates chr16:635517-635774 and C16orf13 in exon with genomic coordinates chr16:635611-635774.

6. The colorectal cancer subtype identifier according to any one of claim 3, wherein for the identification of CMS3 at least one exon skipping event is present selected from C16orf13 in exon with genomic coordinates chr16:635280-635340, C16orf13 in exon with genomic coordinates chr16:635517-635774, C16orf13 in exon with genomic coordinates chr16:635611-635774, AURKA in exon with genomic coordinates chr20:56388686-56388784, EPB41L3 in exon with genomic coordinates chr18:5394676-5394793, KALRN in exon with genomic coordinates chr3:124637207-124637303, ADAM15 in exon with genomic coordinates chr1:155061903-155061975, KRAS in exon with genomic coordinates chr12:25215436-25215560, SLC39A14 in exon with genomic coordinates chr8:22412036-22412206, MYO9A in exon with genomic coordinates chr15:71951776-71951896, TPM1 in exon with genomic coordinates chr15:63044026-63044152, TPM1 in exon with genomic coordinates chr15:63061197-63061273, TPM1 in exon with genomic coordinates chr15:63061712-63061788, MYO6 in exon with genomic coordinates chr6:75894813-75894840, MYO6 in exon with genomic coordinates chr6:75898372-75898411, KIAA1217 in exon with genomic coordinates chr10:24494499-24494604, KIAA1217 in exon with genomic coordinates chr10:24542692-24542770, KIAA1217 in exon with genomic coordinates chr10:24542882-24544481, GIT2 in exon with genomic coordinates chr12:109945259-109945349, MYL6 in exon with genomic coordinates chr12:56160625-56160670, CTNND1 in exon with genomic coordinates chr11:57789036-57789155, CTNND1 in exon with genomic coordinates chr11:57791384-57791673, CTNND1 in exon with genomic coordinates chr11:57791491-57791673, CD44 in exon with genomic coordinates chr11:35208104-35208206, CD44 in exon with genomic coordinates chr11:35211245-35211449, APBB2 in exon with genomic coordinates chr4:40935076-40935139, SEC31A in exon with genomic coordinates chr4:82830936-82830975, SEC31A in exon with genomic coordinates chr4:82830936-82830975, SEC31A in exon with genomic coordinates chr4:82842139-82842481, SEC31A in exon with genomic coordinates chr4:82842184-82842481, SYTL2 in exon with genomic coordinates chr11:85717482-85717530, MYOF in exon with genomic coordinates chr10:93392916-93392955, NAV2 in exon with genomic coordinates chr11:20051288-20051333, GAB1 in exon with genomic coordinates chr4:143434087-143434168, PLEKHM2 in exon with genomic coordinates chr1:15721328-15721388, CLSTN1 in exon with genomic coordinates chr1:9737497-9737554, CLSTN1 in exon with genomic coordinates chr1:9756480-9756510, GOLGA4 in exon with genomic coordinates chr3:37361242-37361305, PBX1 in exon with genomic coordinates chr1:164820071-164820184, FKBP14 in exon with genomic coordinates chr7:30020212-30020304 and XPO1 in exon with genomic coordinates chr2:61525269-61525333.

7. The colorectal cancer subtype identifier according to claim 3, wherein for the identification of CMS4 at least one exon skipping event is present selected from MYO9A in exon with genomic coordinates chr15:71951776-71951896, TPM1 in exon with genomic coordinates chr15:63044026-63044152, TPM1 in exon with genomic coordinates chr15:63061197-63061273, TPM1 in exon with genomic coordinates chr15:63061712-63061788, MYO6 in exon with genomic coordinates chr6:75894813-75894840, MYO6 in exon with genomic coordinates chr6:75898372-75898411, KIAA1217 in exon with genomic coordinates chr10:24494499-24494604, KIAA1217 in exon with genomic coordinates chr10:24542692-24542770, KIAA1217 in exon with genomic coordinates chr10:24542882-24544481, GIT2 in exon with genomic coordinates chr12:109945259-109945349, MYL6 in exon with genomic coordinates chr12:56160625-56160670, BPTF in exon with genomic coordinates chr17:67875555-67875744, MYH11 in exon with genomic coordinates chr16:15708802-15708841, NUMB in exon with genomic coordinates chr14:73279280-73279424, TEAD1 in exon with genomic coordinates chr11:12878888-12878900, SPAG9 in exon with genomic coordinates chr17:50975862-50975901, FAT1 in exon with genomic coordinates chr4:186590367-186590403, TNS1 in exon with genomic coordinates chr2:217830366-217830390, ESYT2 in exon with genomic coordinates chr7:158752780-158752843, SLMAP in exon with genomic coordinates chr3:57925844-57925934, AKAP9 in exon with genomic coordinates chr7:91992157-91992211, RUBCN in exon with genomic coordinates chr3:197691073-197691148, ATP2B4 in exon with genomic coordinates chr1:203733222-203733400, LRRFIP2 in exon with genomic coordinates chr3:37091466-37091538, TBC1D23 in exon with genomic coordinates chr3:100311832-100311877, EHBP1 in exon with genomic coordinates chr2:62987930-62988038, SLK in exon with genomic coordinates chr10:104010815-104010908, WDFY3 in exon with genomic coordinates chr4:84726860-84726911, SMARCC2 in exon with genomic coordinates chr12:56164302-56164368, KIF13A in exon with genomic coordinates chr6:17771113-17771218, MPRIP in exon with genomic coordinates chr17:17180606-17180669, LRRFIP1 in exon with genomic coordinates chr2:237769625-237769818, NIN in exon with genomic coordinates chr14:50756491-50758630, RPS24 in exon with genomic coordinates chr10:78040203-78040225, ACTN1 in exon with genomic coordinates chr14:68878988-68879069, CTNND1 in exon with genomic coordinates chr11:57789036-57789155, CTNND1 in exon with genomic coordinates chr11:57791384-57791673, CTNND1 in exon with genomic coordinates chr11:57791491-57791673, CD44 in exon with genomic coordinates chr11:35208104-35208206, CD44 in exon with genomic coordinates chr11:35211245-35211449, APBB2 in exon with genomic coordinates chr4:40935076-40935139, SEC31A in exon with genomic coordinates chr4:82830936-82830975, SEC31A in exon with genomic coordinates chr4:82830936-82830975, SEC31A in exon with genomic coordinates chr4:82842139-82842481, SEC31A in exon with genomic coordinates chr4:82842184-82842481, SYTL2 in exon with genomic coordinates chr11:85717482-85717530, MYOF in exon with genomic coordinates chr10:93392916-93392955, NAV2 in exon with genomic coordinates chr11:20051288-20051333, GAB1 in exon with genomic coordinates chr4:143434087-143434168, PLEKHM2 in exon with genomic coordinates chr1:15721328-15721388, CLSTN1 in exon with genomic coordinates chr1:9737497-9737554, CLSTN1 in exon with genomic coordinates chr1:9756480-9756510, GOLGA4 in exon with genomic coordinates chr3:37361242-37361305, PBX1 in exon with genomic coordinates chr1:164820071-164820184 and FKBP14 in exon with genomic coordinates chr7:30020212-30020304.

8. The colorectal cancer subtype identifier according to claim 3, wherein for distinction of CMS2 and CMS3 from CMS1 and CMS4, at least one exon skipping event is present selected from FCGRT in exon with genomic coordinates chr19:49521694-49521813, DOCK6 in exon with genomic coordinates chr19:11229297-11229390, EXOC7 in exon with genomic coordinates chr17:76090328-76090397, BPTF in exon with genomic coordinates chr17:67875555-67875744, PXN in exon with genomic coordinates chr12:120224642-120224727, EXOC1 in exon with genomic coordinates chr4:55888887-55888932, PLOD2 in exon with genomic coordinates chr3:146077861-146077924, CD47 in exon with genomic coordinates chr3:108049618-108049651, CD47 in exon with genomic coordinates chr3:108050577-108050602, ARHGEF11 in exon with genomic coordinates chr1:156938417-156938513, PBRM1 in exon with genomic coordinates chr3:52558248-52558413, MAGI1 in exon with genomic coordinates chr3:65448021-65448057, MBNL1 in exon with genomic coordinates chr3:152446703-152446757, TNC in exon with genomic coordinates chr9:115064646-115064919, ENAH in exon with genomic coordinates chr1:225504990-225505053, BAZ2B in exon with genomic coordinates chr2:159397073-159397100, RAI14 in exon with genomic coordinates chr5:34813573-34813660, FNIP1 in exon with genomic coordinates chr5:131710577-131710661, MAP3K7 in exon with genomic coordinates chr6:90544551-90544632, ERBIN in exon with genomic coordinates chr5:66068876-66069020, SULF2 in exon with genomic coordinates chr20:47659398-47659452, SORBS1 in exon with genomic coordinates chr10:95414493-95414655 and SORBS1 in exon with genomic coordinates chr10:95414493-95414862.

9. The colorectal cancer subtype identifier according to claim 3, wherein for distinction of CMS2 and CMS4 from CMS1 and CMS3, at least one exon skipping event is present selected from SRSF6 in exon with genomic coordinates chr20:43459152-43459420, WASH3P in exon with genomic coordinates chr15:101972595-101972694, OPA1 in exon with genomic coordinates chr3:193626091-193626202, GOLGB1 in exon with genomic coordinates chr3:121719645-121719753, GOLGB1 in exon with genomic coordinates chr3:121719645-121719768, APLP2 in exon with genomic coordinates chr11:130137255-130137291, CPNE1 in exon with genomic coordinates chr20:35658929-35659014, WIPF1 in exon with genomic coordinates chr2:174597600-174597858, ATG9A in exon with genomic coordinates chr2:219228433-219228482, NUBP2 in exon with genomic coordinates chr16:1786737-1786955 and ANO1 in exon with genomic coordinates chr11:70111125-70111191.

10. The colorectal cancer subtype identifier according to claim 3, wherein the alternative splicing event is based on at least one exon skipping event on at least one gene selected from ITGAE, CCNDBP1, CPT1B, CDC16, PTPN6, ACCS, EXOSC9, ZNF611, MRRF, NUP153, WARS, D2HGDH, LUC7L, EPB41, DGUOK, MDM4, PTP4A2, MKNK2, CCDC112, FRYL, CEP78, FNBP1, ECT2, ANKRD26, ZMIZ2, C6orf48, USPL1, RBM39, MIS12, AFMID, MACROD1, FN1, WBP1, XPO1, PTPN18, ARHGAP27, C16orf13, FCGRT, DOCK6, EXOC7, BPTF, PXN, EXOC1, PLOD2, CD47, ARHGEF11, PBRM1, MAGI1, MBNL1, TNC, ENAH, BAZ2B, RAI14, FNIP1, MAP3K7, ERBIN, SULF2, SORBS1, SRSF6, WASH3P, OPA1, GOLGB1, APLP2, CPNE1, WIPF1, ATG9A, NUBP2, ANO1, AURKA, EPB41L3, KALRN, ADAM15, KRAS, SLC39A14, MYO9A, TPM1, MYO6, KIAA1217, GIT2, MYL6, MYH11, NUMB, TEAD1, SPAG9, FAT1, TNS1, ESYT2, SLMAP, AKAP9, RUBCN, ATP2B4, LRRFIP2, TBC1D23, EHBP1, SLK, WDFY3, SMARCC2, KIF13A, MPRIP, LRRFIP1, NIN, RPS24, ACTN1, CTNND1, CD44, APBB2, SEC31A, SYTL2, MYOF, NAV2, GAB1, PLEKHM2, CLSTN1, GOLGA4, PBX1 and FKBP14.

11. A method for predicting the outcome for a subject suffering from colorectal cancer using PSI values based on the occurrence of at least one alternative splicing event comprising the steps:
g) collecting a sample from a tumor of a subject having cancer;
h) determining PSI values;
i) converting PSI values to determine the disease outcome indication.

12. The method of claim 11, wherein the alternative splicing event is at least one exon skipping event.

13. The method of claim 12, wherein the at least one exon skipping event is at least one exon skipping event on at least one gene selected from ITGAE, CCNDBP1, CPT1B, CDC16, PTPN6, ACCS, EXOSC9, ZNF611, MRRF, NUP153, WARS, D2HGDH, LUC7L, EPB41, DGUOK, MDM4, PTP4A2, MKNK2, CCDC112, FRYL, CEP78, FNBP1, ECT2, ANKRD26, ZMIZ2, C6orf48, USPL1, RBM39, MIS12, AFMID, MACROD1, FN1, WBP1, XPO1, PTPN18, ARHGAP27, C16orf13, FCGRT, DOCK6, EXOC7, BPTF, PXN, EXOC1, PLOD2, CD47, ARHGEF11, PBRM1, MAGI1, MBNL1, TNC, ENAH, BAZ2B, RAI14, FNIP1, MAP3K7, ERBIN, SULF2, SORBS1, SRSF6, WASH3P, OPA1, GOLGB1, APLP2, CPNE1, WIPF1, ATG9A, NUBP2, ANO1, AURKA, EPB41L3, KALRN, ADAM15, KRAS, SLC39A14, MYO9A, TPM1, MYO6, KIAA1217, GIT2, MYL6, MYH11, NUMB, TEAD1, SPAG9, FAT1, TNS1, ESYT2, SLMAP, AKAP9, RUBCN, ATP2B4, LRRFIP2, TBC1D23, EHBP1, SLK, WDFY3, SMARCC2, KIF13A, MPRIP, LRRFIP1, NIN, RPS24, ACTN1, CTNND1, CD44, APBB2, SEC31A, SYTL2, MYOF, NAV2, GAB1, PLEKHM2, CLSTN1, GOLGA4, PBX1 and FKBP14.

14. The method of claim 11, wherein the conversion of PSI values to determine the disease outcome indication classifies the tumors as CMS1, CMS2, CMS3 or CMS4.

15. The method of claim 11, wherein the conversion of PSI values to determine the disease outcome indication is used to decide, whether the subject is subjected to a treatment by anti-tumor therapy and/or surveillance of progress of the disease.

16. A kit for predicting the outcome for a subject suffering from colorectal cancer using PSI values based on the occurrence of at least one alternative splicing event, preferably at least one exon skipping event on at least one gene, selected from ITGAE, CCNDBP1, CPT1B, CDC16, PTPN6, ACCS, EXOSC9, ZNF611, MRRF, NUP153, WARS, D2HGDH, LUC7L, EPB41, DGUOK, MDM4, PTP4A2, MKNK2, CCDC112, FRYL, CEP78, FNBP1, ECT2, ANKRD26, ZMIZ2, C6orf48, USPL1, RBM39, MIS12, AFMID, MACROD1, FN1, WBP1, XPO1, PTPN18, ARHGAP27, C16orf13, FCGRT, DOCK6, EXOC7, BPTF, PXN, EXOC1, PLOD2, CD47, ARHGEF11, PBRM1, MAGI1, MBNL1, TNC, ENAH, BAZ2B, RAI14, FNIP1, MAP3K7, ERBIN, SULF2, SORBS1, SRSF6, WASH3P, OPA1, GOLGB1, APLP2, CPNE1, WIPF1, ATG9A, NUBP2, ANO1, AURKA, EPB41L3, KALRN, ADAM15, KRAS, SLC39A14, MYO9A, TPM1, MYO6, KIAA1217, GIT2, MYL6, MYH11, NUMB, TEAD1, SPAG9, FAT1, TNS1, ESYT2, SLMAP, AKAP9, RUBCN, ATP2B4, LRRFIP2, TBC1D23, EHBP1, SLK, WDFY3, SMARCC2, KIF13A, MPRIP, LRRFIP1, NIN, RPS24, ACTN1, CTNND1, CD44, APBB2, SEC31A, SYTL2, MYOF, NAV2, GAB1, PLEKHM2, CLSTN1, GOLGA4, PBX1 and FKBP14, comprising means for determining the PSI value.

17. A method for treating a subject suffering from colorectal cancer using PSI values based on the occurrence of alternative splicing comprising the steps:
j) collecting a sample from a tumor of a subject having cancer;
k) determining PSI values;
l) conversion of PSI values to determine the disease outcome indication.

18. The method according to claim 17, wherein the conversion of PSI values to determine the disease outcome indication classifies the tumors as CMS1, CMS2, CMS3 or CMS4.

19. The method according to claim 11, wherein the subject is a human.

20. The method according to 17, wherein the subject is a human
